(19) United States
(12) Patent Application Publication
(10) Pub. No.: US 2010/0004390 A1
(43) Pub. Date: Jan. 7, 2010

(54) BIODEGRADABLE METAL-CHELATING POLYMERS AND VACCINES

(75) Inventors: William G. Turnell, Del Mar, CA (US); Zaza D. Gomurashvill, La Jolla, CA (US); Benjamin W. Parcher, San Diego, CA (US); Jonathan Hughes, Carlsbad, CA (US); Jeffrey N. Anderl, San Diego, CA (US)

Correspondence Address: DLA PIPER LLP (US), 4365 EXECUTIVE DRIVE, SUITE 1100, SAN DIEGO, CA, 92121-2133, US

(73) Assignee: MediVas, LLC, San Diego, CA (US)

(21) Appl. No.: 12/437,435

(22) Filed: May 7, 2009

Related U.S. Application Data
(60) Provisional application No. 61/051,270, filed on May 7, 2008.

(51) Int. Cl. A61K 47/48; C08G 69/44; C08L 77/12

(52) U.S. Cl. 525/54.1; 528/291; 525/420

(57) ABSTRACT

The invention provides metal-chelating poly(ether amide) polymers useful in preparation of polymer compositions for delivering a variety of cargo molecules, such as bioactive agents. In solution metal ions and cargo molecules, such as vaccine epitopes, that include metal avid amino acids can be loaded into the polymer compositions and held in a non-covalent complex. Nanoparticles of such polymer compositions can also be prepared directly from the solution.

### RELATED APPLICATIONS

This application claims priority under 35 U.S.C. §119(e) of U.S. Provisional application Ser. No. 61/051,270, filed May 7, 2008 which is hereby incorporated by reference in its entirety.

### FIELD OF THE INVENTION

Polyaminocarboxylic acids are frequently used as complexing or chelating agents in the decontamination of living organisms and recently have been proposed as substitutes for phosphates in detergents. These compounds are known to form complexes with various metal ions, most frequently with trivalent lanthanides. Polyaminocarboxylic acids, such as EDTA (ethylenediaminetetraacetic acid) and DTPA (diethylenetriamine-pentaacetic acid), are also commonly used to chelate diagnostic and therapeutic moieties to an in vivo delivery composition.

Polymers with complexing properties also have been created. The clinical application of macromolecular gadolinium (Gd) complexes as MRI contrast agents has been reported. For example, Gd chelates have been conjugated to biomedical polymers, including linear poly(amino acids), polysaccharides, proteins and various dendrimers. Co-polymerization of DTPA anhydride with diamines and complexation with Gd(III) also has been reported. However, clinical application of such macromolecular systems, including those prepared from typical biodegradable polymers, such as dextrans, polylysine, and the like, has been limited by the slow excretion of Gd [III] complexes and consequent long-term tissue accumulation of toxic Gd ions. Therefore, despite these advances in the art, a need exists for more and better biodegradable macromolecular systems that avoid the problem of slow excretion.

### SUMMARY OF THE INVENTION

The present invention provides a composition comprising at least one polymer or a salt thereof selected from:

a PEA polymer having a chemical formula described by general structural formula (I),

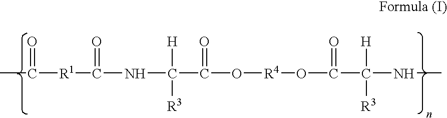

wherein n ranges from about 15 to about 150;

R¹ is independently from —CH₂—N(CH₂CO₂H)—R⁶—N(CH₂CO₂H)—CH₂— or a structure of formula (II), and combinations thereof; wherein R⁶ is independently selected from the group consisting of (C₂-C₁₂) alkylene, p-C₆H₄, (C₂-C₄)alkyloxy (C₂-C₄)alkylene, and CH₂CH₂N(CH₂CO₂H)CH₂CH₂; and wherein, R⁷ in formula (II) is selected from hydrogen, (C₁-C₁₂) alkyl, and a protective group;

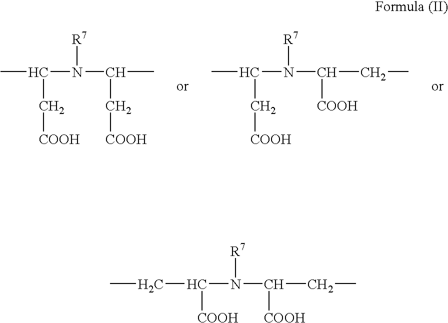

R³s in individual n units are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, —(CH₂)₂SCH₃, CH₂OH, CH(OH)CH₃, (CH₂)₄NH₃⁺, (CH₂)₃NHC(═NH₂⁺)NH₂, 4-methylene imidazolinium, CH₂COO⁻, (CH₂)₂COO⁻ and combinations thereof;

R⁴ is independently selected from the group consisting of (C₂-C₂₀) alkylene, (C₂-C₂₀) alkenylene, (C₂-C₆) alkyloxy (C₂-C₁₂) alkylene, CH₂CH(OH)CH₂, CH₂CH(CH₂OH), a bicyclic-fragment of a 1,4:3,6-dianhydrohexitol of structural formula (III), a fragment of 1,4-anhydroerythritol, and combinations thereof;

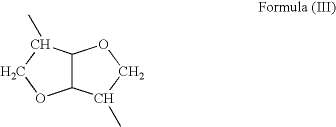

or a PEA polymer having a chemical formula described by structural formula (IV):

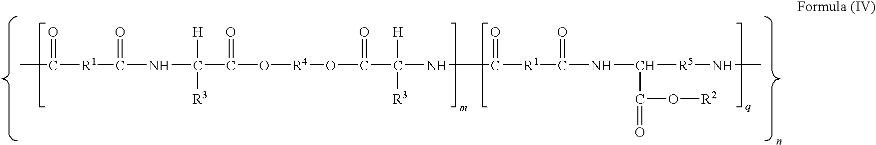

wherein n ranges from about 15 to about 150, m ranges about 0.1 to 0.9; p ranges from about 0.9 to 0.1; and wherein

R¹ is —CH₂—N(CH₂CO₂H)—R⁶—N(CH₂CO₂H)—CH₂—, wherein R⁶ is independently selected from the group consisting of (C₂-C₁₂) alkylene, p-C₆H₄, (C₂-C₄)alkyloxy (C₂-C₄)alkylene, CH₂CH₂N(CH₂CO₂H)CH₂CH₂, and a structure of formula (II), wherein, R⁷ is selected from hydrogen, (C₁-C₁₂) alkyl, a protective group, and combinations thereof;

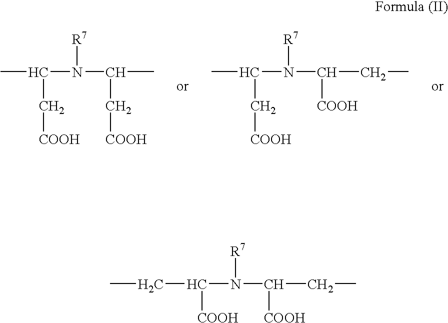

R² is independently selected from the group consisting of hydrogen, (C₁-C₁₂) alkyl or (C₆-C₁₀) aryl and a protective group;

R³s in individual n units are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, —(CH₂)₂SCH₃, CH₂OH, CH(OH)CH₃, (CH₂)₄NH₃⁺, (CH₂)₃NHC(═NH₂⁺)NH₂, 4-methylene imidazolinium, CH₂COO⁻, (CH₂)₂COO⁻ and combinations thereof; R⁴ is independently selected from the group consisting of (C₂-C₂₀) alkylene, (C₂-C₂₀) alkenylene, (C₂-C₆) alkyloxy (C₂-C₁₂) alkylene, CH₂CH(OH)CH₂, CH₂CH(CH₂OH), a bicyclic-fragment of a 1,4:3,6-dianhydrohexitol of structural formula (III), a fragment of 1,4-anhydroerythritol, and combinations thereof; and R⁵ is independently selected from the group consisting of (C₂-C₄) alkyl.

In another embodiment the invention provides methods for making nanoparticles by contacting together 1) at least one polymer having a chemical structure described by Formula (I) or (IV) dissolved in aqueous solution; and 2) a metal ion selected from the group consisting of Ca²⁺, Mg²⁺, Mn²⁺, Co²⁺, Fe²⁺ and Fe³⁺, Zn²⁺, Ni²⁺; so as to form nanoparticles containing a non-covalent complex of the polymer and the transition metal ion.

In still another embodiment, the invention provides methods for delivering a cargo molecule to a subject by administering to the subject an invention composition.

In yet another embodiment, the invention provides methods for making nanoparticles by

a) contacting together in an aqueous solution under polycondensation conditions:

1) an invention chelating polymer of Formula (I) or (IV);

2) a metal ion selected from the group consisting of Ca²⁺, Mg²⁺, Mn²⁺, Co²⁺, Fe²⁺ and Fe³⁺ Zn²⁺, Ni²⁺ and Gd³⁺; and

3) an aprotic polar solvent;

b) forming nanoparticles containing a non-covalent complex of the polymer and the metal cation in the solution; and
c) obtaining the nanoparticles from the solution by size exclusion separation.

### A BRIEF DESCRIPTION OF THE FIGURES

FIG. 1 is a representation of the ¹H-NMR spectrum of polymer: PEA EDTA-Leu(6), (Formula Ia).

FIG. 2 is a graph showing survival curve of immunized mice after infection with influenza virus. Filled ⋄=animals immunized with buffer only; ▴=animals immunized intraperitonally with virus, positive control; stars=animals immunized intranasally once with both the HAPR8 ectodomain and NPPR8, formulated with PEA EDTA-Leu(6)-Zn and Poly I:C; ▪=mice immunized intranasally with HAPR8 ectodomain and NPPR8 formulated with PEA EDTA-Leu(6)-Zn).

FIG. 3 is a graph showing weight change of immunized mice after infection with influenza virus. ∘=animals immunized with buffer only; stars=animals immunized intraperitonally with virus, positive control; ▴=average weight change for animals immunized once intranasally with the HAPR8 ectodomain and NPPR8 formulated with PEA EDTA-Leu(6)-Zn and Poly I:C; ▪=mice immunized intranasally with HAPR8 ectodomain and NPPR8 formulated with PEA EDTA-Leu(6)-Zn; ⋄=animals immunized intranasally with HAPR8 ectodomain formulated with PEA EDTA-Leu(6)-Zn.

FIG. 4 is a graph showing average percentage weight change in immunized mice after infection with influenza virus. ▪=weight change of animals immunized with PEA EDTA-Leu(6) polymer in formulation buffer (All mice died of viral infection by day 7); ∘=mice immunized intraperitonally with virus, positive control; ▴=average weight change for animals intranasally administered HAPR8-3 and NPPR8 with PEA EDTA-Leu(6)-Zn and Poly I:C particles (One mouse, dead by day 8, produced no measurable antibody response to HA protein); Δ=mice immunized subcutaneously with HAPR8-3 and NPPR8 with PEA EDTA-Leu(6)-Zn and Poly I:C particles (All but one mouse died by day 8).

FIG. 5 is the amino acid sequence of His-tagged nucleoprotein from Influenza Strain A/PR/8/34 (Mount Sinai) (SEQ ID NO:1).

FIG. 6 is the amino acid sequence of HAPR8Ectodomain antigen from Influenza Strain A/PR/8/34 (Mount Sinai) (SEQ ID NO:2).

FIG. 7 is the amino acid sequence of HAPR8-2 His-tagged subfragment antigen of HA protein from Influenza Strain A/PR/8/34 (Mount Sinai). The underlined portion is appended as a signal sequence for bacterial expression and does not appear in the amino acid sequence produced by the bacterium (SEQ ID NO:3).

FIG. 8 is the amino acid sequence of HAPR8 3 His-tagged subfragment antigen of HA protein from Influenza Strain A/PR/8/34 (Mount Sinai). The underlined portion is appended as a signal sequence for bacterial expression and does not appear in the amino acid sequence produced by the bacterium (SEQ ID NO:4).

FIG. 9 is the amino acid sequence of the His-tagged nucleoprotein antigen from Influenza Strain A/VN/1203/2004 (SEQ ID NO:5).

FIG. 10 is the amino acid sequence of HAVN ectodomain antigen from Influenza Strain A/VN/1203/2004 (SEQ ID NO:6).

FIG. 11 is the amino acid sequence of HAVN-2 His-tagged subfragment of HA protein from Influenza Strain A/VN/1203/2004. The underlined sequence is appended as a signal sequence for bacterial expression and does not appear in the amino acid sequence produced by the bacterium (SEQ ID NO:7).

FIG. 12 is the amino acid sequence of HAVN-3 His-tagged subfragment antigen of HA protein from Influenza Strain A/VN/1203/2004. The underlined sequence is appended as a signal sequence for bacterial expression and does not appear in the amino acid sequence produced by the bacterium (SEQ ID NO:8).

### A DETAILED DESCRIPTION OF THE INVENTION

The present invention is based on the discovery that biodegradable metal-chelating polymers can be obtained by incorporation of polyaminocarboxylic acids into backbone of poly(ester amides) PEAs. Such biodegradable metal-chelating polymers will chelate metal cations without binding of a separate metal affinity ligand.

The invention biodegradable metal-chelating polymers are related structurally to known poly(ester amides) PEAs, except that in the present invention the di-acid building block used in solution polycondensation of known PEAs has been replaced with a poly-acid of the EDTA type (i.e. a polyaminoacetic acid). The monomer prepared from this type polyamino acid for use in synthesis of the invention polymers is the equivalent dianhydride, which under the conditions for solution condensation interacts with diamine to form amide bonds with bis(alpha-amino acyl)-diol diester monomers. Thus, during polymerization, two carboxylic acid groups of the polyaminoacetic acid are taken up in formation of the polymer backbone, which bears iminoacetic groups therealong. Remaining unbound carboxylic acid groups of in-line residues of the polyaminoacetic acids in the polymer are free to chelate metal cations in a solution.

Accordingly, in one embodiment the invention provides a composition comprising at least one polymer or a salt thereof selected from:

a polymer having a chemical formula described by general structural formula (I),

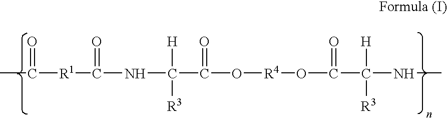

wherein n ranges from about 15 to about 150;

R¹ is independently from —CH₂—N(CH₂CO₂H)—R⁶—N(CH₂CO₂H)—CH₂—, wherein R⁶ is independently selected from the group consisting of (C₂-C₁₂) alkylene, p-C₆H₄, (C₂-C₄)alkyloxy (C₂-C₄)alkylene, and CH₂CH₂N(CH₂CO₂H)CH₂CH₂, or a structure of formula (II), wherein, R⁷ is selected the group consisting of hydrogen, (C₁-C₁₂) alkyl, and a protective group, and combinations thereof, and;

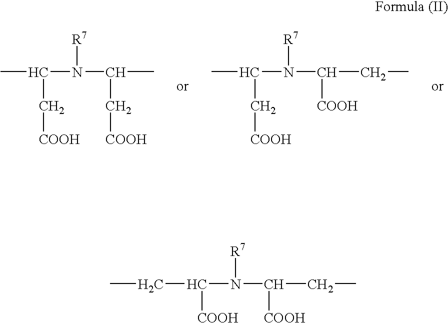

R³s in individual n units are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, —(CH₂)₂SCH₃, CH₂OH, CH(OH)CH₃, (CH₂)₄NH₃⁺, (CH₂)₃NHC(═NH₂⁺)NH₂, 4-methylene imidazolinium, CH₂COO⁻, (CH₂)₂COO⁻ and combinations thereof;

R⁴ is independently selected from (C₂-C₆) alkyloxy (C₂-C₁₂) alkylene, (C₂-C₂₀) alkylene, (C₂-C₂₀) alkenylene, CH₂CH(OH)CH₂, CH₂CH(CH₂OH), a bicyclic-fragment of a 1,4:3,6-dianhydrohexitol of structural formula (III), a fragment of 1,4-anhydroerythritol, and

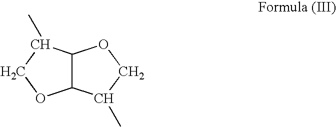

or a PEA polymer having a chemical formula described by structural formula (IV):

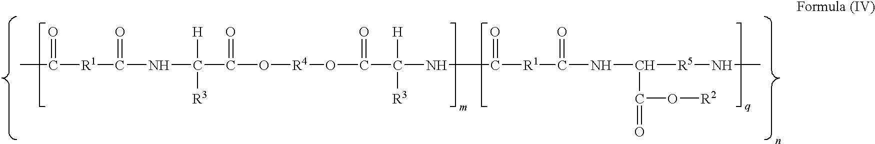

wherein n ranges from about 15 to about 150, m ranges about 0.1 to 0.9; p ranges from about 0.9 to 0.1; and wherein

R¹ is —CH₂—N(CH₂CO₂H)—R⁶—N(CH₂CO₂H)—CH₂—, wherein R⁶ is independently selected from the group consisting of (C₂-C₁₂) alkylene, p-C₆H₄, (C₂-C₄)alkyloxy (C₂-C₄)alkylene, CH₂CH₂N(CH₂CO₂H)CH₂CH₂, and a structure of formula (II), wherein, R⁷ is selected from hydrogen, (C₁-C₁₂) alkyl, a protective group, and combinations thereof;

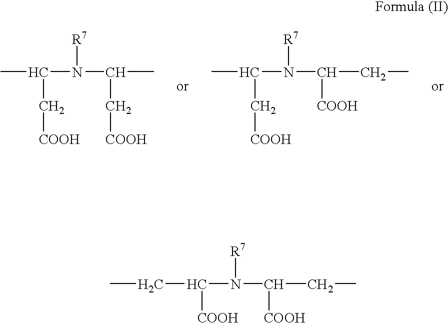

R² is independently selected from the group consisting of hydrogen, (C₁-C₁₂) alkyl or (C₆-C₁₀) aryl and a protective group;

R³s in individual n units are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, —(CH₂)₂SCH₃, CH₂OH, CH(OH)CH₃, (CH₂)₄NH₃⁺, (CH₂)₃NHC(═NH₂⁺)NH₂, 4-methylene imidazolinium, CH₂COO⁻, (CH₂)₂COO⁻ and combinations thereof; R⁴ is independently selected from the group consisting of (C₂-C₂₀) alkylene, (C₂-C₂₀) alkenylene, (C₂-C₆) alkyloxy (C₂-C₁₂) alkylene, CH₂CH(OH)CH₂, CH₂CH(CH₂OH), a bicyclic-fragment of a 1,4:3,6-dianhydrohexitol of structural formula (III), a fragment of 1,4-anhydroerythritol, and combinations thereof; and

R⁵ is independently selected from the group consisting of (C₂-C₄) alkyl.

The invention metal-chelating polymers are biodegradable and can be water soluble. The invention metal-chelating polymers may have counter-ions associated therewith, for example Na and K counter ions, to form a salt.

Additionally, when the polymer is synthesized using iminodisuccinic acid (Formula II), the invention metal-chelating polymer of formula (I) or (IV) may contain imide units, as a product of cyclodehydration of polyamic acid. Then invention polymer will include chemical structures as shown in Formula (V):

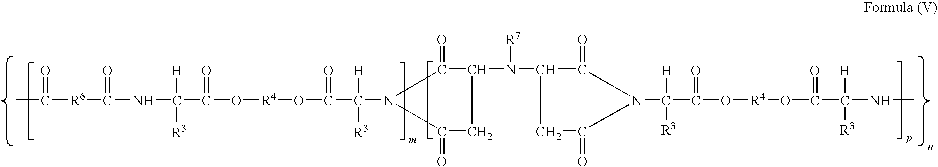

The invention metal-chelating polymers optionally can be associated with counter-ions selected from the group consisting of sodium and potassium. For example, the polymer can be associated with sodium ion to increase water solubility of the polymer or of a composition containing the invention metal-chelating polymer. Invention polymers can be stored in the free acid form or as a metal salt, such as an alkali metal salt. Protons in pendant imminoacetic acid groups can be partially or fully displaced with Na or K ions to form salts.

As used herein, the term “aryl” refers to structural formulae herein to denote a phenyl radical or an ortho-fused bicyclic carbocyclic radical having about nine to ten ring atoms in which at least one ring is aromatic. In certain embodiments, one or more of the ring atoms can be substituted with one or more of nitro, cyano, halo, trifluoromethyl, or trifluoromethoxy. Examples of aryl include, but are not limited to, phenyl, naphthyl, and nitrophenyl. As used herein, the term “alkenylene” refers to structural formulae herein to mean a divalent branched or unbranched hydrocarbon chain containing at least one unsaturated bond in the main chain or in a side chain.

As used herein, the term “alkenyl” refers to straight or branched chain hydrocarbyl groups having one or more carbon-carbon double bonds.

As used herein, “alkynyl” refers to straight or branched chain hydrocarbyl groups having at least one carbon-carbon triple bond.

As used herein, “aryl” refers to aromatic groups having in the range of 6 up to 14 carbon atoms.

The metal-chelating polymers used in the invention compositions are poly-condensates. The ratios “m” and “p” in Formula (IV and V) are defined as irrational numbers in the description of these poly-condensate polymers. Moreover, as “m” and “p” will each take up a range within any polycondensate, such a range cannot be defined by a pair of integers. Each polymer chain is a string of monomer residues linked together by the rule that all bis-amino acyl diol-diester (i) and a directional amino acid (e.g. lysine) monomer residues (ii) are linked either to themselves or to each other by a polyamino acid monomer residue (iii). Thus, only linear combinations of i-iii-i; i-iii-ii (or ii-iii-i) and ii-iii-ii are formed. In turn, each of these combinations is linked either to themselves or to each other by a diacid monomer residue (iii) for PEA Each polymer chain is therefore a statistical, but non-random, string of monomer residues composed of integer numbers of monomers, i, ii and iii. However, in general, for polymer chains of any practical average molecular weight (i.e., sufficient mean length), the ratios of monomer residues “m” and “p” in formulas (IV) will not be whole numbers (rational integers). Furthermore, for the condensate of all poly-dispersed copolymer chains, the numbers of monomers i, ii and iii averaged over all of the chains (i.e. normalized to the average chain length) will not be integers. It follows that the ratios can only take irrational values (i.e., any real number that is not a rational number). Irrational numbers, as the term is used herein, are derived from ratios that are not of the form n/j, where n and j are integers.

As used herein, the terms “amino acid” and “α-amino acid” mean a chemical compound containing an amino group, a carboxyl group and a pendent R group, such as the R³ groups defined herein. As used herein, the term “biological α-amino acid” means the amino acid(s) used in synthesis are selected from phenylalanine, leucine, glycine, alanine, valine, isoleucine, methionine, or a mixture thereof. As used herein, the term “adirectional amino acid” means a chemical moiety within the polymer chain obtained from an α-amino acid, such that the R group (for example R⁵ in Formulas (IV) is inserted within the polymer backbone.

The invention metal-chelating polymers can be prepared as solution polycondensation products of polyaminoacetic acid-derived bisanhydridrides with diamines, specifically bis(alpha amino acyl)-diol diesters in aprotic solvents. Ester bonds inherent in bis(alpha-aminoacyl)-diester monomers and their derived polymers can be hydrolyzed by bioenzymes, forming non toxic degradation products.

In one alternative, at least one of the α-amino acids used in fabrication of the invention metal-chelating polymers is a biological α-amino acid. For example, when the R³s are CH₂Ph, the biological α-amino acid used in synthesis is L-phenylalanine. In alternatives wherein the R³s are CH₂CH(CH₃)₂, the polymer contains the biological α-amino acid, L-leucine. By varying the R³s within monomers as described herein, other biological α-amino acids can also be used, e.g., glycine (when the R³s are H), alanine (when the R³s are CH₃), valine (when the R³s are CH(CH₃)₂), isoleucine (when the R³s are CH(CH₃)CH₂CH₃), phenylalanine (when the R³s are CH₂C₆H₅), or methionine (when the R³s are —(CH₂)₂SCH₃, and combinations thereof. In yet another alternative embodiment, all of the various α-amino acids contained in the polymers used in making the invention OEG-based polymer delivery compositions are biological α-amino acids, as described herein.

In yet another embodiment, the invention provides methods for delivering one or more cargo agents to a site in the body of a subject. In this embodiment, the invention methods involve injecting into an in vivo site in the body of the subject an invention composition that has been formulated as a dispersion of polymer nanoparticles wherein at least one cargo molecule is held in a coordination complex with a metal ion therein. The injected nanoparticles will slowly release the complexed cargo molecules.

A dispersion of the invention nanoparticles can be injected parenterally, for example subcutaneously, intramuscularly, or into an interior body site, such as an organ. The biodegradable nanoparticles act as a carrier for the at least one, for example two different cargo molecules, into the circulation for targeted and timed release systemically. Invention polymer particles in the size range of about 10 nm to about 500 nm will enter directly into the circulation for such purposes

The biodegradable polymers used in the invention composition can be designed to tailor the rate of biodegradation of the polymer to result in continuous delivery of the cargo molecule over a selected period of time, depending upon the choice of the building blocks of the polymer, the metal cation and, particularly, the polyamino acids included in the invention composition.

Suitable protecting groups for use in the PEA metal-chelating polymers include a tosyl salt (e.g. Tos-OH), or another as is known in the art. Suitable 1,4:3,6-dianhydrohexitols of general formula (III) include those derived from sugar alcohols, such as D-glucitol, D-mannitol, or L-iditol. Dianhydrosorbitol is the presently preferred bicyclic fragment of a 1,4:3,6-dianhydrohexitol for use in fabrication of the invention OEG-based polymer delivery compositions.

In one alternative, R³ is CH₂Ph and the α-amino acid used in synthesis is L-phenylalanine. In alternatives wherein R³ is CH₂—CH(CH₃)₂, the polymer contains the α-amino acid, leucine. By varying R³, other α-amino acids can also be used, e.g., glycine (when R³ is H), alanine (when R³ is CH₃), valine (when R³ is CH(CH₃)₂), isoleucine (when R³ is CH(CH₃)—CH₂—CH₃), phenylalanine (when R³ is CH₂—C₆H₅), lysine (when R³ is —(CH₂)₄—NH₂); or methionine (when R³ is —(CH₂)₂SCH₃).

Choice of the in-line α-amino acid (by selection of R³s) and the diol used in fabrication of the bis-(L-leucine)-1,6-hexanediol diester monomer (designated as Leu(6)) as well as the in-line poly acetic acid residue in an invention polymer aid in determination of the electronic properties of the invention metal-chelating polymer. For example, the polymer designated herein Leu(6)-EDTA is composed of alternating hydrophobic segments (i.e., Leu(6)) and strongly charged segments (i.e., in-line EDTA). The resulting polymer is water soluble. Metal-chelation at a mol fraction of 1:1 (metal:inline-EDTA) neutralizes the in-line EDTA groups and so the metallated polymer becomes a string of alternating hydrophobic segments and neutral polar segments. The resulting metallated polymer readily condenses into particles using the invention methods (capturing as it does so any pre-mixed cargo molecule with metal-binding properties).

The amino acid residue in the bis(α-amino acid)-diol diester segment of the invention polymer, in addition to conferring biodegradability and biocompatibility, can be selected to impart different biophysical and biochemical properties to the metal-bound, otherwise neutral, polar polymer. For example, by substituting Arg or Lys for Leu in the foregoing example to create Arg(6)- or Lys(6)EDTA, the invention polymer is composed of alternating positively charged segments and negatively charged segments, and is thus charge-neutral and polar overall. Such a polymer will interact weakly with poly(nucleic acids), which are themselves strongly negatively charged. However, upon metal-chelation, the negatively-charged in-line EDTA segments are neutralized, resulting in a cationic polymer, which will interact strongly with poly(nucleic acids) both via the Culombic interaction of the positively charged Arg(6) segments with the negatively charged poly(nucleic acid) and via the metal-mediated ionic bonds between the metallated in-line EDTA segments and the poly(nucleic acid). Thus, in this example, substitution of Arg or Lys for Leu in the invention polymer described above is sufficient to confer greater stability, where required, in the loading of negatively charged, polar cargo molecules.

Conversely, substitution of Asp or Glu for Leu in the Leu(6)-EDTA example above renders the invention polymer most suitable for loading of cationic, polar cargo molecules. Substitution of Ser, Thr, Asn, Gln, and combinations thereof for Leu in the Leu(6)-EDTA example above renders the invention metallated polymers most suitable for loading of neutral, polar, or poly(hydroxylated) cargo molecules, such as sugars and heavily glycosylated proteins.

In addition to the selection of the in-line α-amino acid residue to tailor the invention metallated polymer to the properties of a particular cargo molecule, the diol of the bis-AA(diol) segment can be selected to confer different polymer chain flexibilities (T_{g}) and thereby different particle mechanical properties, as well as different polymer chain solubilities. For example, rigid bicyclic dianhydrohexitole diol (isosorbide, DAS) results in a water-insoluble polymer (formula Ib); whereas shorter aliphatic diol or hydrophilic 1,4-anhydroerythritol imparts hydrophilicity and water solubility to the polymer (formula Ic).

Accordingly, Co-polymers X—Y—X—Z, in which Y and Z are exchangeable statistically can be fabricated in which X is an in-line chelating segment and Y and Z are different bis-AA(diol) segments, allowing fine tuning of the polymer to one or more cargo molecules.

Non-limiting examples of polyamino acids useful in fabrication of the invention metal-chelating polymers include Diethylenetriamine pentaacetic acid (DTPA), Nitrilotriacetic acid (NTA), ethylenediamine tetraacetic acid (EDTA), ethylene glycol-bis(2-aminoethylether)-N,N,N′,N′-tetraacetic acid (EGTA), iminodiacetic acid (IDA, and the like. Synthesis of dianhydride residues of such polyamino acids is illustrated in the Examples herein. Dianhydrides of DTPA and EDTA are commercially available.

Aprotic polar solvents, such as N,N-dimethylaacetamide (DMAC), dimethyl sulfoxide (DMSO) and N-methyl-2-pyrrolidone (NMP) are used in formation of invention metal-chelating polymers from solution polycondensation of dianhydride with diamine. Depending on the molecular structures and hydrophobicities of the diamine and dianhydride used during polycondensation, the obtained polymers are either soluble in aqueous solution or hydrophobic (and therefore, insoluble).

Due to the iminoacetic groups along the polymer backbone, the invention polymers can form a coordination complex with various metal cations. Transition metal cations useful for forming a metal coordination complex with the invention metal-binding polymers to form an invention “metallated polymer” include, but are not limited to, those of Ca, Mg, Mn, Ni, Co, Fe (both 2⁺ and 3⁺), and Zn. Of the non-radioactive and non-imaging metals, the most important on bio-safety grounds is Zn, followed by Ni. Metal ions useful in preparation of a radioactive or imaging metallated polymer include radioactive metal isotopes such as Rhenium, iridium, and Yttrium. In one embodiment, the transition metal cation bound to the invention polymer presently preferred for imaging in diagnostic applications is Gd(III) and the poly amino acid used in fabrication of the invention metal-chelating polymer is DTPA.

Because free iminoacetic groups are located along the flexible polymer chains used in the invention compositions and methods, the metal ion can be arranged in the best position relative to the binding sites on the surface of cargo molecule(s). As a result, the cargo molecule(s) can be bound non-covalently, to the polymer via the metal affinity complex formed. In other embodiments, the free —NH₂ ends of the polymer molecule can be acylated to assure that the cargo molecule will attach only via a metal affinity complex and not to the free ends of the polymer.

A transition metal cation bound in a coordination complex to the iminoacetic acid groups of the invention metal-chelating polymers creates a composition, referred to herein as a “metallated polymer”, in which at least one free valency of chelated metal is available to bind a therapeutic cargo molecule that has affinity for the metal cations. As described more fully below, the amino acids in the polymer backbone further contribute to the sum of electrical forces that stabilize the cargo molecule in the metallated polymer compositions and in nanoparticles of such compositions.

Suitable cargo molecules that can be complexed by invention metallated polymers include polar bioactive agents, such as drugs; “biologics”, and His-tagged molecules. A “biologic” as the term is used herein encompasses natural and synthetically produced proteins, peptides, polyamino acids, fusion proteins, and poly nucleic acids, including vaccine antigens, such as those described herein as SEQ ID NOS: 1-8. A “macromolecular biologic” as the term is used herein includes biologics whose bioactivity depends upon a unique three-dimensional folded structure of the molecule, such as proteins, polypeptides and polynucleic acids. It has also been discovered that bioactivity of vaccine antigens also depends upon preservation in the vaccine formulation of the natural three-dimensional folded structure of the molecule as it occurs in the parent pathogen It has been discovered that the electric forces in the invention metallated polymers can capture from aqueous solution and stabilize biologics and macromolecular biologics as well as lipophilic cargo molecules containing micro-regions of negative polarity, as described more fully hereinbelow.

The existence of at least one Histidine residue in a biologic cargo molecule (e.g, protein, peptidic antigen, or fusion construct with His tag) is an important factor contributing to binding of the cargo biologic to the polymer. A His at the amino- or carboxyl-terminus of the cargo biologic (i.e., a His-tag) results in improved specificity of binding of the cargo molecule to the metal ion in the metal affinity complex. Therefore, in one embodiment, at least one to about 10 adjacent His residues, for example, about six His residues (i.e, a “hexa-His tag”), are incorporated at one or both of the amino- and carboxy-termini as a tag to ensure binding efficiency. If a His tag is added, the His tag and the metal chelate, for example the Ni or Zn-metal chelate, are allowed to remain in the final composition, e.g., the nanoparticles.

Since the pK value of the histidine groups contributing to the binding lies in the neutral range, the binding of a cargo biologic molecule to the polymer might be expected to occur at a pH value of about 7. However, the actual pK value of an individual amino acid can vary strongly depending on the influence of neighboring amino acid residues. Various experiments have shown that, depending on the protein structure, the pK value of an amino acid can deviate from the theoretical pK value up to one pH unit. Therefore, a reaction solution with a pH value of about 8 often achieves an improved binding.

Other metal binding amino acids, such as cysteine and tryptophane, present in a cargo biologic molecule also contribute to the metal binding. Moreover, it is not necessary that a biologic belong to the class of established metal-binding proteins to be suitable for use as a biologic cargo molecule in the invention compositions and methods. Crystallographers routinely use transition metal-bound analogs of a protein under structural investigation as an essential part of the structure-solving process. This procedure is called the “isomorphous replacement method” and has resulted in the discovery that all proteins and polynucleic acids bind transition metals at least weakly, irrespective of whether a metal-binding site(s) is biologically functional or not in the molecule (M Babor et al. Proteins (2008) 70:208-217 and Supplement found on the worldwide web at interscience.wiley.com/jpages/0887-3585/suppmat/ and N Valls et al. J. Biol Inorg Chem (2005) 10:476-482).

In the present invention, it has been discovered that the weak affinity of all biologics, including macromolecular biologics, for transition metals and the backbone amino acids of the invention compositions is sufficient to capture and hold such cargo molecules in invention metallated polymers and in nanoparticles made using invention polycondensation methods. The avidity afforded by invention metallated polymers stabilizes the loaded particles. Surprisingly, it has been discovered that even certain bioactive molecules that are lipophilic as macromolecules can be chelated by an invention metallated polymer. Such bioactive molecules are characterized by having a cLogP in the range from about 2.0 to 6.0, but also are characterized by the presence of micro-regions of negative polarity consisting of 1) unsaturated regions (including aromatic groups) and 2) lone pairs of electrons as in O- and S- and N-containing groups. Invention metallated polymers having complexed such lipophilic cargo molecules can also be formulated as nanoparticles using the invention methods for polycondensation of nanoparticles. Examples of such macromolecularly lipophilic drug compounds presently preferred for complexing by the invention metallated polymers include, but are not confined to, Taxanes, such as Paclitaxel and Docetaxel, and limus compounds, such as Sirolimus, Everolimus, and Biolimus.

More particularly, paclitaxel has a cLogP of about 3.5 so it has the macro properties of a highly lipophilic drug with a very low aqueous solubility. However an inspection of its surface at the atomic level shows that, while the molecule is hydrophobic on a macromolecular level, nonetheless there are micro-regions of polarity provided by aromatic groups and by oxygen atoms. These micro-regions of polarity found over the surface of the hydrophobic molecule account for binding of paclitaxel to a cavity in its target protein (beta-tubulin) that is lined with polar as well as with hydrophobic amino acid side-chains. It is believed that the avidity of such compounds (i.e., the sum of micro-affinities) for the weakly binding free coordination sites in the invention metallated polymers leads to stabilization of lipophilic cargo molecules within the nanoparticles of invention metallated polymers.

As another example, Rapamycin (Sirolimus), one of the most hydrophobic drugs in current use, has a cLogP of about 5.5 and so is about 100-fold more hydrophobic than Paclitaxel. Yet Rapamycin bears several micro-regions of either unsaturated bonds (akin to the aromatic regions on Paclitaxel) or lone pairs of electrons around oxygen atoms (as in Paclitaxel). It is believed that these micro-electronic regions are important at the molecular level in directing the specificity of Rapamycin affinity for its protein biotarget, mTOR. Because they represent concentrated sources of strong, multivalent ionic bonds, metal ions are ideally suited to seek out and lock onto micro-polar regions to be found on even the most hydrophobic of clinically useful compounds, for example, compounds that in vivo bind specifically to a ligand site in a larger target protein.

Another example of a cargo molecule suitable for loading in the invention metallated polymers is Serum albumin (SA), which is commercially available and well recognized in the field. SA has the following chemical and biological properties that make it particularly suited for inclusion in a metal-chelating polymer coating, implant or particles (as shown in Example 5 herein): 1) a native high-affinity metal-binding site, 2) incidental targeting property for angiogenic blood vessels around tumors; and 3) high blood compatibility (creating the potential that SA-loaded particles could be used for intravenous delivery).

Due to its high blood compatibility, when used as a cargo molecule in an invention composition, SA can have several therapeutic uses: 1) as a detoxification agent for metals, 2) as a detoxification agent for lipophilic (and therefore cell-penetrating) toxins (for example, a plant defense molecule such as Paclitaxel), 3) as a plasma transport agent for native hydrophobic. molecules (fatty acids, steroids), or 4) as an agent for maintenance of osmotic pressure of the blood (vital for the regulation of the exchange of blood volume with other bodily fluids).

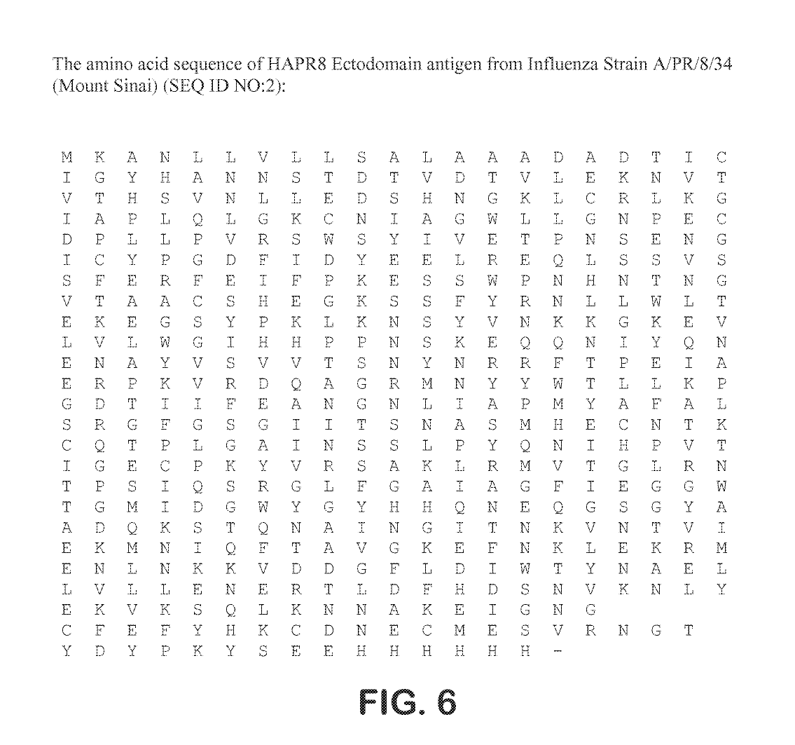

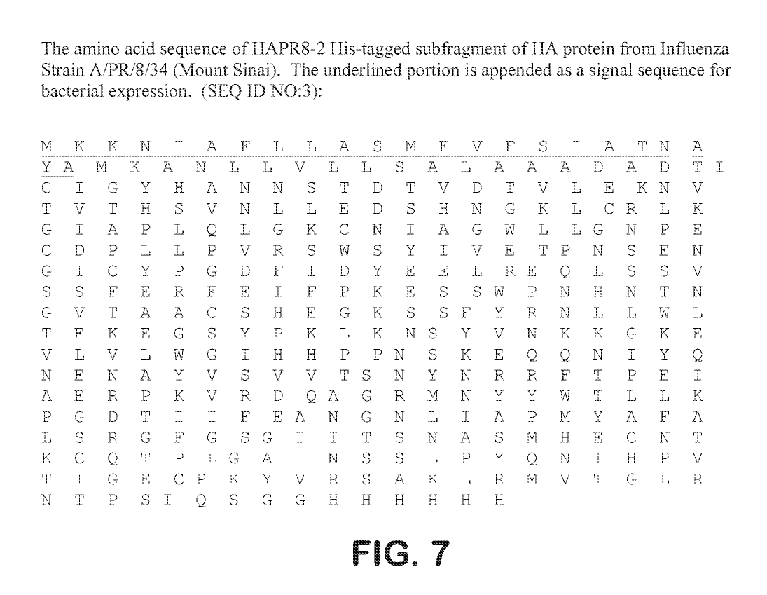

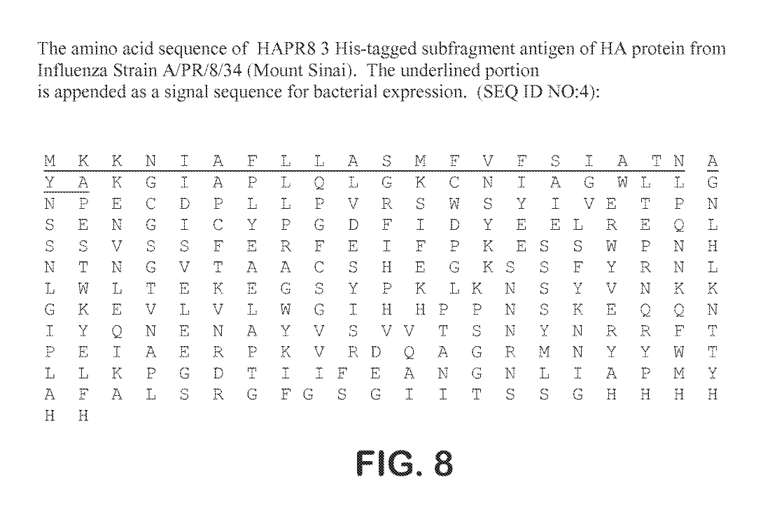

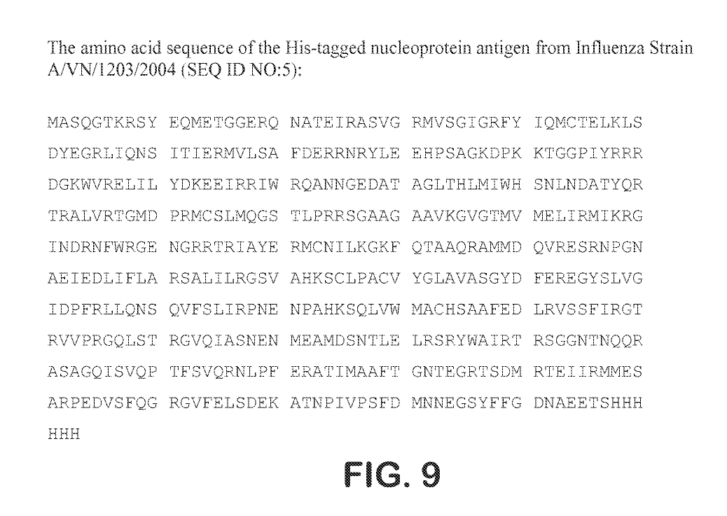

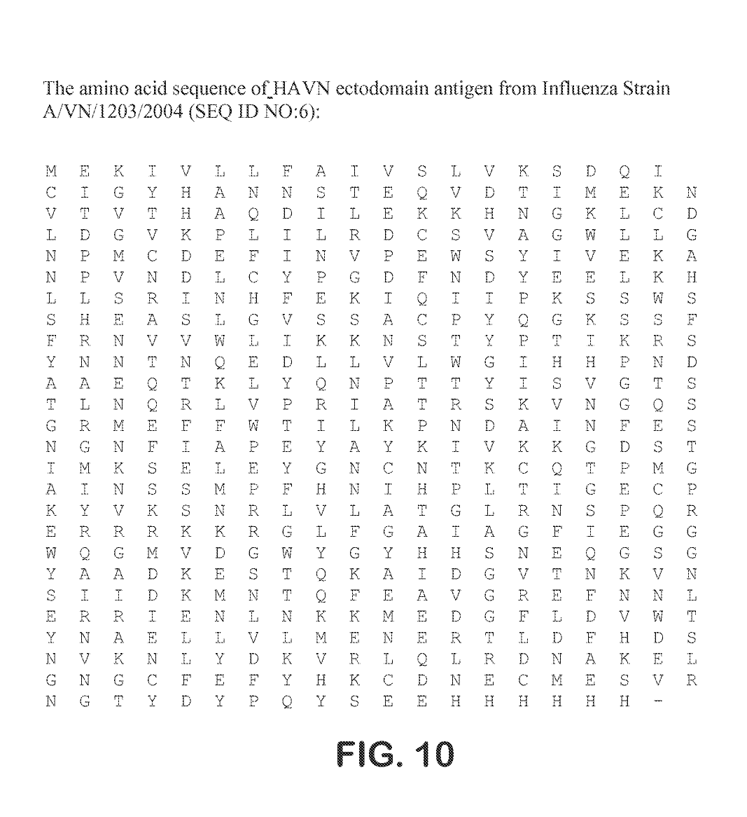

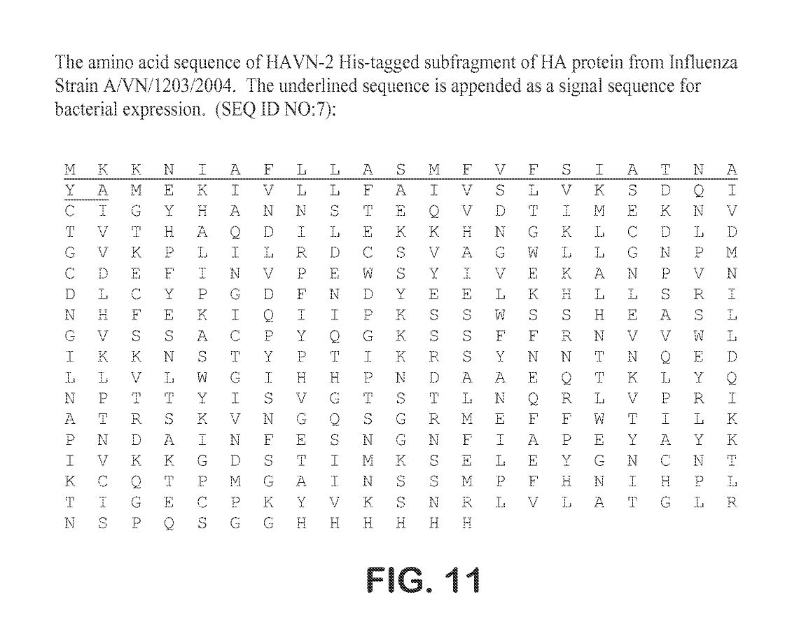

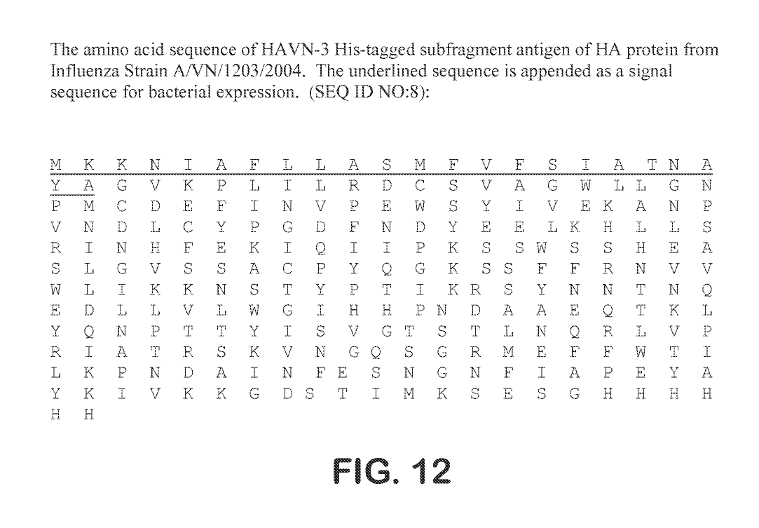

Further specific examples of cargo bioactive agents that are suitable for chelating with the invention metallated polymers include, without limitation, drugs, therapeutic biologics, such as Insulin, Human growth hormone, and Calcitonin; therapeutic and targeting antibodies, and active fragments thereof, known therapeutic Blood factors, such as clotting factors, and both protein and glycoprotein antigens, such as those suitable for inclusion in subunit vaccines. Additionally, peptides, (including. those containing pathogenic epitopes for subunit vaccines) can be incorporated into the invention metallated polymer compositions. In particular amino acid sequences comprising a pathogenic epitope can be incorporated into invention metallated polymer compositions in formulation of a subunit vaccine in which the unique three-dimensional folded structure of the epitope is preserved. Non-limiting examples of such antigenic amino acid sequences include those described herein as SEQ ID NOS: 1-8 in FIGS. 5-12 herein.

Formulations of cargo-loaded metallated polymers are various and include implants, coatings and nanoparticles, such as vaccine formulations. For example, in one embodiment, the invention provides methods for formulating the invention metallated polymers as nanoparticles using a technique of solution polycondensation, which avoids the need for emulsion technology as is commonly used in formation of polymer particles. The invention metallated polymers, whether additionally complexed with one or more cargo molecules, or not, are readily formulated into nanoparticles as a final step in the polycondensation of the metallated polymers, as described in Examples 4 and 5 herein. Furthermore, the invention polycondensation methods result in particles that are more dispersive in aqueous environment than particles based upon the more hydrophobic first generation of PEAs wherein the diol used in fabrication is an aliphatic dicarboxylic acid, as disclosed in Chu C C, Katsarava R, U.S. Pat. No. 6,503,538 B1.

In brief, the invention method for preparation of nanoparticles of cargo-loaded metallated polymers involves the following steps: a) Preparing a homogenous mixture of cargo molecule and aqueous solution of an invention polymer; b) Preparing a cargo molecule /transition metal salt solution by bolus addition of aqueous metal salt to a stirred solution of the cargo molecule; and c) generating nanoparticles by drop-wise addition of the solution of a) into b) under stirring at room temperature. Nanoparticles are recovered from the reaction solution by size-exclusion filtration, dialysis, or centrifugation and washing techniques, for example as is known in the art and described herein in Examples 4 and 5.

Alternatively, the invention metallated polymers with chelated cargo molecule(s) can be applied as a viscous liquid coating to the exterior of various types of particles using various techniques known in the art, such as spraying, dipping, and the like. For use as a coating, cargo molecule(s) for inclusion in the invention are selected from, but are not confined to, blood factors, including serum albumin, transferrin, antibodies and active fragments thereof, as well as His-tagged fusion constructs of such cargo molecules. Such coatings also can be applied to at least a portion of the exterior of various types of solid objects used in medical treatments, as is known in the art. Such a coating may be used to enhance the blood or tissue compatibility of the particles or medical devices to which the coating is applied.

In another embodiment, the invention metal-chelating polymers without chelated metal cations can be administered to a subject for the purpose of metal detoxification and/or wound care, being formulated for administration as an implant or as particles, either alone or as an adjuvant accompanying a therapeutic bioactive agent.

In still another embodiment, the invention metallated polymers can be formulated as coatings, implants and particles to be used for presentation and/or delivery of therapeutic drugs and biologics. For example, invention metallated polymers can be co-loaded with drug and a biologic ligand, such as an antibody or other ligand targeting a cell surface marker, specific receptor or protein docking site, wherein the biologic ligand is used to deliver the composition and chelated drug to a target cell or cell type, such as a type of cancer cell. The drug can be selected to kill, to block docking of a native ligand molecule, or to prevent replication of a molecule in the target tissue or cancer cell.

In yet another embodiment of the invention, particles of the invention polymers are co-loaded with a cargo paramagnetic or ferromagnetic metal, as described herein, and a biologic ligand. The paramagnetic or ferromagnetic metal is used for diagnostic imaging of a target organ, tissue or cell to which the biologic ligand delivers the composition, once injected parenterally. Methods of using such diagnostic compositions are well known in the art.

In still another embodiment, a radioactive metal, as is described herein, is chelated by the invention metal-chelating polymer and the second molecule, a targeting ligand as is known in the art and described herein, is used for tissue or cell targeting. For example a radioactive metal can be targeted to stem cells in a cancerous tumor to kill the stem cells by incorporating a ligand, such as an antibody that binds specifically to a cell surface marker thereon, for example an antibody that binds specifically to CD20.

In another embodiment of the invention, nanoparticles for diagnostic imaging are co-loaded with a diagnostic metal ion as described herein, (e.g. Gd³⁺) and a ligand that binds specifically to a target cell, organ or tissue. Methods for conducting, Gd imaging are well known in the art and include, but are not limited to, in vivo magnetic resonance imaging (MRI) in which the diagnostic composition is injected parenterally for diagnostic imaging and the targeting ligand, as is known in the art and described herein, is used for tissue or cell targeting.

Consequently, in one embodiment, the invention metal-chelating polymers are chelated with diagnostic metals to form a diagnostic composition that can be administered in vivo for use in imaging a desired target cell, organ or tissue, yet the polymer composition is readily biodegraded and excreted. Thus, invention diagnostic compositions made using the invention metal-chelating polymers avoid long-term tissue accumulation of chelated toxic ions and can be formulated as nanoparticles using methods of polycondensation described herein.

In still other embodiment, invention polymers are conjugated to bioactive agents via polymer end groups and or end-group conjugation is used to obtain ABA type block-systems, where B is a polymer of Formula (I) or Formula (IV) and the A block is selected from such compounds as PEG (oligo- or polyethyleneglycol), polysaccharides, lipids, biologic macromolecules such polypeptides or poly(nucleic acids) and active agents. In both cases it is preferable that the B block polymer macrochain possess equal amounts or numbers of active end-groups, either amine or anhydride (other conjugation sites will be pendent carboxylic groups along the macrochain).

Synthesis of a B block for incorporation into a ABA block chelating polymer with equal amounts or numbers of identical end groups was achieved by using an imbalance technique, wherein one difunctional monomer used in polycondensation of invention chelating polymers as described herein (i.e., either a diamine, or activated polyacid) was introduced with pre-calculated excess, at the beginning of polymerization. The process became complicated when the anhydride end groups were used in excess because large amounts of polymeric rings (macrocycles) were generated as monitored by Maldi-TOF spectroscopy. However, it has been discovered that introduction of inorganic base (e.g., K₂CO₃) significantly decreases the reaction rate and allows better control of Mw of resultant linear ABA block polymer.

Invention chelating polymer molecules may have a bioactive agent attached thereto via end-group conjugation, optionally via a linker. For example, in one embodiment, the chelating polymer is contained in a polymer-bioactive agent end-group conjugate having structural formula VIII:

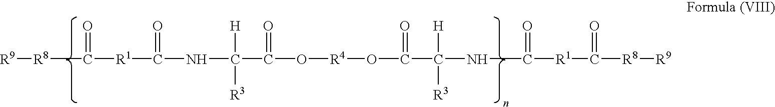

Wherein n, R¹, R³ and R⁴ are as above, R⁸ is selected from the group consisting of —O—, —S—, and NR¹⁰, wherein R¹⁰ is H or (C₁-C₈) alkyl; and R⁹ is a bioactive agent as described herein.

To obtain vaccine formulations, in one embodiment, an amino acid sequence comprising at least one pathogenic epitope that maintains its native conformation is attached to the invention chelating polymer via unbound carboxylic acid groups of in-line residues of the polyaminoacetic acids in the invention polymer (i.e., in the R³s in invention chelating polymer or metallated polymer. Alternatively in vaccine formulation, unbound carboxylic acid groups of in-line residues of the polyaminoacetic acids in the invention polymer are free to chelate metal cations in solution to form a metallated polymer. The metal cations facilitate further attachment of metal-binding amino acids in pathogenic epitopes. Nanoparticles of the metallated polymer vaccine formulation are readily obtained directly from the polymer-containing solution without the need for emulsion technology as is commonly used in formation of polymer particles. Methods for vaccine formulation as nanoparticles using invention chelating (e.g. metallated) polymers are described herein in Examples 8 and 9.

In yet another embodiment, which is described in detail below, end-group conjugated R⁹ of Formula (VIII) is a bioactive agent, such one or more of the various immunostimulating adjuvants. Immunostimulating adjuvants include drugs, such as Imiquimod; a lipid, such as QS-21; a nucleic acid, such as the dsRNA analog Polyl:PolyC; or an immunostimulatory protein, such as GM-CSF. Particularly desirable immunostimulating adjuvants useful for end-group conjugation to an invention polymer enhance the effectiveness of invention chelating polymers formulated as vaccine compositions are arranged by type in Table 6 below.

TABLE 6

| ADJUVANT Name | Type |
|---|---|
| Calcitrol | Drug |
| Imiquimod | Drug |
| Loxoribine | Drug |
| Poly rA: Poly rU | Drug |
| S-28463 | |
| SM360320 | Drug |
| Adjumer | Polymer |
| CRL1005 | Polymer |
| PLGA, PGA & PLA | Polymer |
| Pluronic L121 | Polymer |
| PMMA | Polymer |
| PODDS | Polymer |
| SAF-1 | Polymer |
| SPT | Polymer |
| Avridine | Lipid |
| Bay R1005 | Lipid |
| DDA | Lipid |
| DHEA | Lipid |
| DMPC | Lipid |
| DMPG | Lipid |
| D-Murapalmitine | Lipid |
| DOC/Alum Complex | Lipid |
| ISCOM | Lipid |
| Iscoprep 7.0.3 | Lipid |
| Liposomes | Lipid |
| MF59 | Lipid |
| Montanide ISA 51 | Lipid |
| Montanide ISA 720 | Lipid |
| Murapalmitine | Lipid |
| Non-Ionic Surfactant Vesicles | Lipid |
| Polysorbate 80 | Lipid |
| Protein Cochleates | Lipid |
| Span 85 | Lipid |
| Stearyl Tyrosine | Lipid |
| Theramide | Lipid |
| Gerbu Adjuvant | Lipid/Sugar |
| QS-21 | Lipid/Sugar |
| Quil A | Lipid/Sugar |
| Walter Reed Liposomes | Lipid/Salt |
| Algal Glucan | Sugar |
| Algammulin | Sugar |
| Gamma Inulin | Sugar |
| GMDP | Sugar |
| ImmTher | Sugar |
| Murametide | Sugar |
| Pleuran | Sugar |
| Threonyl-MDP | Sugar |
| Adju-Phos | Salt |
| Alhydrogel | Salt |
| Calcium Phosphate Gel | Salt |
| Rehydragel HPA | Salt |
| Rehydragel LV | Salt |
| Cytokine-containing liposomes | Biologic |
| GM-CSF | Biologic |
| Immunoliposomes Containing Antibodies | Biologic |
| to Costimulatory Molecules (DRV) | |
| Single stranded DNA | Biologic |
| Single stranded RNA | Biologic |
| Double stranded DNA | Biologic |
| Double stranded RNA | Biologic |
| Interferon-γ | Biologic |
| Interleukin-12 | Biologic |
| Interleukin-1B | Biologic |
| Interleukin-2 | Biologic |
| Interleukin-7 | Biologic |
| LT-OA (LT-Oral ADJUVANT) | Biologic |
| Sclaro Peptide | Biologic |
| Sendai Proteoliposomes, Sendai- | Biologic |
| containing Lipid Matrices | |
| Ty Particles | Biologic |
| Squalane | Oil |

An example of the method for end-group conjugation of an immunostimulating adjuvant in preparation of nanoparticles of a vaccine formulation is illustrated in Example 10 herein.

Alternatively still, as shown in structural formula (IX) below, a linker, —X—Y—, can be inserted between R⁸ and bioactive agent R⁹, in the molecule of structural formula (I) and (IV), wherein X is selected from the group consisting of (C₁-C₁₈) alkylene, (C₂-C₈) alkyloxy (C₂-C₂₀) alkylene, substituted alkylene, (C₃-C₈) cycloalkylene, substituted cycloalkylene, 5-6 membered heterocyclic system containing 1-3 heteroatoms selected from the group O, N, and S, substituted heterocyclic, (C₂-C₁₈) alkenyl, substituted alkenyl, alkynyl, substituted alkynyl, C₆ and C₁₀ aryl, substituted aryl, heteroaryl, substituted heteroaryl, alkylaryl, substituted alkylaryl, arylalkynyl, substituted arylalkynyl, arylalkenyl, substituted arylalkenyl, arylalkynyl, substituted arylalkynyl and wherein the substituents are selected from the group H, F, Cl, Br, I, (C₁-C₆) alkyl, —CN, —NO₂, —OH, —O(C₁-C₄) alkyl, —S(C₁-C₆) alkyl, —S[(═O)(C₁-C₆) alkyl], —S[(O₂)(C₁-C₆) alkyl], —C[(═O)(C₁-C₆) alkyl], CF₃, —O[(CO)—(C₁-C₆) alkyl], —S(O₂)[N(R¹¹R¹²)], —NH[(C═O)(C₁-C₆) alkyl], —NH(C═O)N(R¹¹R¹²), —N(R¹¹R¹²); where R¹¹ and R¹² are independently H or (C₁-C₆) alkyl; and Y is selected from the group consisting of —O—, —S—, —S—S—, —S(O)—, —S(O₂)—, —NR¹⁰—, —C(═O)—, —OC(═O)—, —C(═O)O—, —OC(—O)NH—, —NR¹⁰C(—O)—, —C(═O)NR¹⁰—, —NR¹⁰C(═O)NR¹⁰—, —NR¹⁰C(═O)NR¹⁰—, and

### —NR¹⁰C(═S)NR¹⁰—.

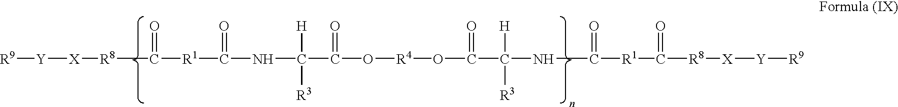

In still another embodiment, invention chelating polymers can be used in design of ABA type block-systems, wherein B is a polymer of Formula (I) or Formula (IV) and the A block is selected from such compounds as PEG (oligo- or polyethyleneglycol), polysaccharides, lipids, biologic macromolecules such polypeptides or poly(nucleic acids) and bioactive agents. The invention ABA block polymers are formed by a technique of end-group conjugation as described in Example 10 herein.

In methods of making invention ABA block polymers that utilize invention chelating or metallated polymers, as well as in all end-group conjugation procedures using such polymers, it is preferable that the B polymer macrochain possess equal active end-groups: either amine or anhydride (other conjugation sites will be pendent carboxylic groups along the macrochain).

Synthesis of invention chelating polymer containing equal amounts or numbers of active end groups is utilized in end-group conjugation whether in simple end group conjugation of bioactive agents, as described above, or in formation of invention ABA block polymers. For both of these procedures, an imbalance technique, wherein one difunctional monomer used in polycondensation of invention chelating polymers as described herein (i.e., either a diamine, or activated polyacid) is introduced with pre-calculated excess, at the beginning of polymerization. The process becomes complicated when the anhydride end group was used in excess because large amounts of polymeric rings (macrocycles) were generated as monitored by Maldi-TOF spectroscopy. However, it has been discovered that introduction of inorganic base (e.g., K₂CO₃) significantly decreases the reaction rate and allows better control of Mw of resultant linear ABA block polymer.

In one embodiment, PEG is introduced as the A block in a ABA block polymer in order to increase solubility of a highly insoluble cargo drug, which is held in a coordination complex by the metallated polymer. The metallated polymer with insoluble cargo drug forms the B block, which is flanked on both sides by the solubility enhancing PEG molecules as the A blocks. It has surprisingly been discovered that in this embodiment of the invention the size of nanoparticles formed from the ABA block polymer is substantially decreased compared to the size of nanoparticles formulated using other embodiments of the invention. For example, nanoparticles of such ABA block polymers have been obtained in the range from about 50 nm to about 100 nm, for example about 68 nm.

The invention is further illustrated by the following non-limiting Examples.

### Example 1

Materials Reagents: Diethylenetriamine pentaacetic dianhydride (DTPA-DA, 98%), ethylenediamine tetraacetic dianhydride (EDTA-DA, 98%), Ethylene glycol-bis(2-aminoethylether)-N,N,N′,N′-tetraacetic acid (EGTA, IDRANAL™ IV), all from Sigma-Aldrich were used as received. Other dianhydrides, for example EGTA dianhydride, can be prepared by acetic anhydride dehydration of the parent tetraacid in pyridine as reported by Geigy, J. R. A.-G. in Fr. Patent 1,548,888 (C1.C07d); Chem. Abstr. (1969) 71:81380q.

Iminodisuccinic acid (IDS) disodium salt (Baypure CX100 G, 77%) was a gift sample from Obermeier GmbH & Co, Bad Berleburg, Germany. Amino acids: L-leucine, L-phenylalanine, glycine, L-arginine, L-lycine and diols 1,3-propanediol and 1,6-hexanediol were obtained from Sigma-Aldrich.

Anhydrous solvents Dimethylformamide (EMD Chemicals, Inc, NJ), N,N-dimethyl formamide (DMF), dimethylsulfoxide (DMSO), N,N-dimethylacetamide (DMAc), (Fisher Scientific) and other solvents Acetone, 2-Propanol, Methanol, Toluene (Spectrum Chemicals, CA) were purchased from commercial sources.

Materials Characterization The chemical structures of monomers and polymer were characterized by standard chemical methods. NMR spectra were recorded by a Bruker AMX-500 spectrometer (Numega R. Labs Inc. San Diego, Calif.) operating at 500 MHz for ¹H NMR spectroscopy. Solvents CDCl₃ or DMSO-d₆ (Cambridge Isotope Laboratories, Inc., Andover, Mass.) were used with tetramethylsilane (TMS) as internal standard.

Melting points of synthesized monomers were determined on an automatic Mettler-Toledo FP62 Melting Point Apparatus (Columbus, Ohio). Thermal properties of synthesized monomers and polymers were characterized on differential scanning calorimeter (DSC) (Mettler-Toledo DSC 822e). Samples were placed in aluminum pans. Measurements were carried out at a scanning rate of 10° C./min under nitrogen flow.

The number and weight average molecular weights (Mw and Mn) and molecular weight distribution (Mw/Mn) of synthesized polymer was determined by Model 515 gel permeation chromatography (Waters Associates Inc. Milford, Mass.) equipped with a high pressure liquid chromatographic pump, a Waters 2414 refractory index detector. Eluent used was 0.1% of LiCl solution in N,N-dimethylacetamide (DMAc) (1.0 mL/min). Two Styragel® HR SE DMF type columns (Waters) were connected and calibrated with polystyrene standards.

Monomer Synthesis:

Synthesis of invention biodegradable polyaminocarboxylic acid-containing polymers involved two basic steps: 1) synthesis of bis-nucleophiles: di-p-toluenesulfonic acid salts of bis(alpha amino acyl)-diol-diesters (compounds of formula VI); and 2) solution polycondensation of the monomer obtained in step 1) with tetracarboxylic acid dianhydrides.

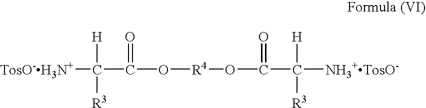

### Synthesis of acid salts of bis(α-amino acid) diesters (general formula VI)

Diesters of structural formula (VI) were prepared using a procedure according to a published procedure: A suspension of an alpha-amino acid (0.1 mol), p-toluenesulfonic acid monohydrate (0.11 mol) and diol (0.05 mol) in 150 mL of toluene was stirred and refluxed in a Dean-Stark condenser, up to evolution of 3.6 mL (0.2 mol) of water (12-24 hours). The heterogenous reaction mixture was cooled down to room temperature and solid products were filtered off, washed with toluene and dried under reduced pressure. Monomers synthesized using this procedure as di-p-toluenesulfoic acid salts are designated herein as follows:

bis-(L-leucyl)-1,6-hexanediol diester, (L-Leu(6)-2TosOH),

bis-(L-phenylalanyl)-1,4:3,6-dianhydrosorbitol diester, (L-Phe(DAS)-2TosOH) bis-(glycine)-1,4-anhydro erythritol diester, (Gly(THF)-2TosOH).

Yields and melting points (Mp) were identical to published data. (Katsarava et al. J. Polym. Sci. Part A: Polym. Chem. (1999) 37:391-407; Z. Gomurashvili et al. J. Macromol. Sci.—Pure. Appl. Chem. (2000) A37:215-227; ZD Gomurashvili et al. US20070282011.)

### Synthesis of bis(L-arginyl)-1,6-hexane diester tetratosyl salt (Arg(6)-4TosOH) of formula (VII)

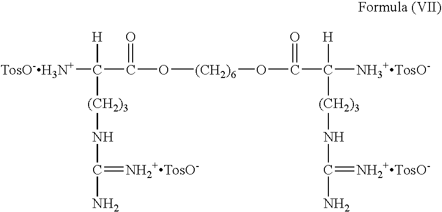

The same procedure as described above was followed for synthesis of monomers having Formula (VII), except that 0.22 mol of p-toluenesulfonic acid monohydrate was employed. For monomer purification, 5 g of crude monomer was dissolved in 30 mL of heated 2-propanol and filtered through filter paper to remove excess of arginine. After storage in a freezer, a viscous monomer layer separated. This procedure was repeated twice and the final product was dried under vacuum over night. Then product was redissolved in 1 g/mL water and freeze-dried. A hygroscopic white material with mp=264-268° C. (DSC, 5° C./min) was collected in 75.9% yield. Elemental analysis: C₄₆H₇₀N₈O₁₆S₄ (1119.35). Calcd.: C, 49.36, H, 6.30, N, 10.01. Found: C, 49.72; H, 6.53; N, 9.96.

### Synthesis of di-p-toluenesulfonic acid salt of bis-L-leucine-PEG₂₀₀-diester, formula (VI), where R³═CH₂—CH(CH₃)₂, R⁴=PEG₂₀₀

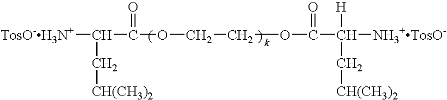

L-leucine (17.46 g) (0.133 mole), 26.53 g (0.14 mole) p-toluenesulfonic acid monohydrate and 11.25 mL (63.4 mmole) of PEG-200 (Aldrich) were suspended in 190 mL dry toluene and stirred using overhead stirrer. Solution heated to reflux for ca. 8 h and evolved water (4.8 mL) was collected in Dean-Stark condenser. After standing at room temperature, brownish-yellow oily layer was separated. Solvent was then decanted off, product was dissolved in 50 mL of 2-propanol and precipitated as oil in 50 mL hexanes. The yield of collected brownish-orange colored crude oily product was 42 g. 10 g of material was redissolved again in hot 150 mL benzene and then allowed to crash out as oil at 4° C. over night. Solvent was decanted and product was dried in vacuum oven at 60° C. for 24 h.

Di-p-toluenesulfonic acid salt of L-leucine-PEG₂₀₀-diester: 500 MHz ¹H NMR (DMSO-d₆, ppm, δ): 0.89 [d, 12H, CH—(CH₃)₂], 1.60 [m, 4H, —CH—CH₂—CH—], 1.74 [m, 2H, —CH—(CH₃)₂], 2.29 [s, 6H, -Ph-CH₃], 3.50 [s, 4H, —OCO—CH₂—CH₂—O—], 3.53-3.64 [m,m ˜10H, —O—CH₂—CH₂—O—]4.00 [s, 2H, ⁺H₃N—CH—], 4.23-4.34 [m,m, 4H, —OCO—CH₂—], 7.14-7.49 [d,d, 8H, Ph], 8.33 [s, 6H, ⁺H₃N—].

### Synthesis of Polymers

Study of the reaction conditions for polycondensation

Polycondensation of EDTA-DA with diamine monomer L-Leu(6).2TosOH was studied in order to optimize the reaction parameters and to increase the Mw of the product.

1.1 Influence of Base

Triethylamine (TEA) was used as a base/catalyst. Reaction of EDTA-DA with 2 molar equivalents of base (1 eq for each tosylate of L-leu(6) comonomer) was compared to reaction with 4 molar equivalents (1 eq for each tosylate and 1 eq for each resulting free carboxylic group formed from EDTA). The results seen in Table 1 below show that, when the carboxylic acids of EDTA are accounted for, use of a two-fold increase in molar equivalents of base more than doubled the size of the polymer in terms of Mw.

TABLE 1
Influence of the Amount of Base on Polymer Molecular Weight (Mw)

| | mol eq. per | | | |
|---|---|---|---|---|
| Base | dianhydride | Mw | MP | PDI |
| TEA | 2 | 33028 | 29484 | 1.44 |
| | 2.2 | 25263 | 23019 | 1.49 |
| | 4 | 71322 | 83906 | 1.97 |
| | 4.04 | 80944 | 89376 | 1.84 |

Solvent: DMF; reaction time: 24 hrs; Temperature: 20° C.; [anhydride] = [diamine] = 0.9M

### 1.2. Influence of Temperature on Polymer MW

During the original PEA EDTA-Leu(6) reaction, which was carried out at 60° C., it was noted that the color of the reaction mixture became noticeably darker, changing from a light yellow to dark amber as well becoming less viscous. To compare temperature effects on color change as well as try to achieve higher Mw, reactions were carried out at 60° C., 40° C., 20° C., and 0° C. The results are listed in Table 2 herein.

As the reaction temperature decreased, the color change of the reaction mixture was less significant and a higher Mw product was obtained. This result suggests that the anhydride is readily reactive with the diamine co-monomer even at low temperatures and that unforeseen side reactions occurred at higher temperatures, which result terminated or inhibited chain extension.

TABLE 2
Influence of the reaction temperature on Mw

| Temperature | | | |
|---|---|---|---|
| (° C.) | Mw | MP | PDI |
| 60 | 25,263 | 23,019 | 1.49 |
| 40 | 33,291 | 31,653 | 1.52 |
| 20 | 71,322 | 83,906 | 1.97 |
| 0 | 143,886 | 104,004 | 2.12 |

Solvent: DMF; reaction time: 24 hrs; [anhydride] = [diamine] = 0.9M

### 3.1.3. Kinetics:

As seen from the above-described polycondensation experiments, the polymer achieved a molecular weight maximum within the first hour. Optimal temperatures for EDTA-diamine condensation reactions (see Table 3 below) were considered to be in the range from 0° C.-20° C.

TABLE 3
Influence of reaction time on Mw

| Reaction | Reaction Time | | | |
|---|---|---|---|---|
| Temperature (° C.) | (hours) | Mw | MP | PDI |
| 0 | 1 | 85362 | 91053 | 1.65 |
| | 3 | 77468 | 86122 | 1.82 |
| | 6 | 80944 | 89376 | 1.84 |
| | 8 | 73501 | 78543 | 1.67 |
| 20 | 1 | 65235 | 80843 | 1.85 |
| | 3 | 71875 | 83906 | 1.97 |
| | 5 | 70312 | 85485 | 1.9 |
| | 8 | 72990 | 88742 | 1.94 |
| | 24 | 71322 | 83906 | 1.97 |
| 40 | 2 | 32685 | 30023 | 1.52 |
| | 20 | 33291 | 31653 | 1.52 |
| 60 | 24 | 25263 | 23019 | 1.49 |

Solvent: DMF; reaction time: 24 hrs; [anhydride] = [diamine] = 0.9M

### 3.1.4. Solvent Choice

Aprotic polar solvents DMSO, DMF and DMAc were compared for suitability in conducting the polycondensation reaction. DMSO was the primary solvent choice because EDTA-dianhydride easily dissolved therein. However, as the polycondensation reaction progressed, the formed polymer became suspended in any of these three reaction solvents. The resulting Mw of polymer obtained in each of the three reaction solvents is shown in Table 3.

Both DMSO and DMF produced discoloration of the polymers and use of DMSO caused a distinct sulfurous odor. Neither drawback was observed when the reaction was conducted in DMAc: the polymer and the suspension were off-white with no odor present. As seen from the data in Table 4, Mw of polymers formed in DMF and DMAc were comparable but use of DMSO resulted in polymer of noticeably lower Mw.

TABLE 4
Influence of the solvent on Mw

| Solvent | Mw | MP | PDI |
|---|---|---|---|
| DMSO | 84722 | 84672 | 1.75 |
| DMF | 143886 | 104004 | 2.12 |
| DMAc | 122825 | 95264 | 2.03 |

Temperature: 0° C.; reaction time: 8 hrs; [anhydride] = [diamine] = 0.9M

### Synthesis of PEA EDTA-Leu(6) Polymer (Formula Ia)

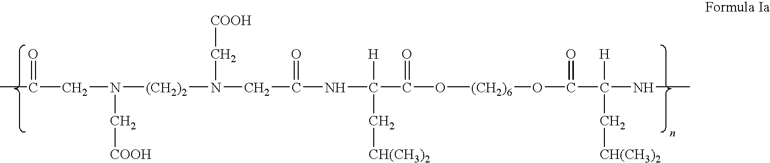

For polycondensation, bis-(L-leucyl)-1,6-hexanediol diester ditosylate (8.32 mmol, 5.734 g) and EDTA-DA (8.32 mmol, 2.133 g) were mixed together followed by the addition of 4.69 mL of dry N,N-dimethylacetamide (DMF) and 4.69 mL of dry triethylamine (TEA) under nitrogen. The reaction was stirred at 0° C. (ice bath) for 8 hrs and quenched by the addition of 5 mol % excess of EDTA-DA (0.42 mmol, 0.107 g). Stirring was continued for an additional 16 hrs at room temperature and the polymer was precipitated in 1 L of acetone (Crude polymer Mw=144,000 g/mol, GPC, DMAc, PS). The supernatant was decanted; polymer was rinsed with acetone, allowed to air dry, then re-suspended in methanol and precipitated in acidified water pH=2 (HCl). The supernatant was decanted and washed thoroughly with deionized (DI) water. The collected polymer was then dried under vacuum at room temperature to a constant weight. The recovered yield product (Formula Ia) was about 60%. The final product after acid work-up had a Mw=50,700 g/mol (GPC, DMAc, PS) and glass transition temperature Tg=77° C.

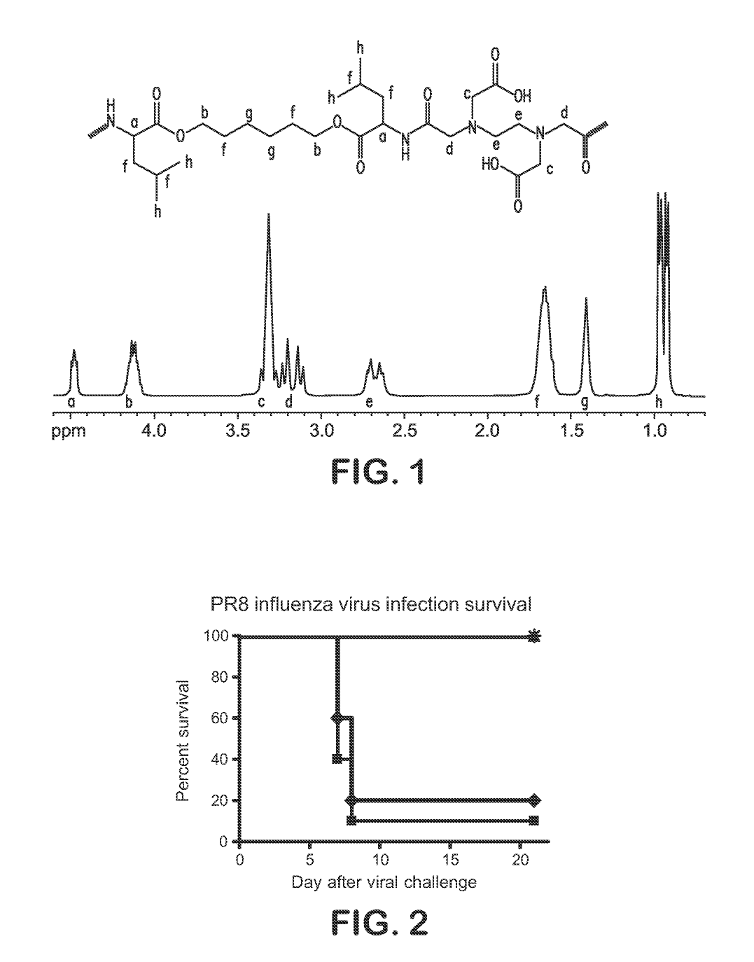

In order to achieve higher solubility in water, this prepared polymer was converted into PEA EDTA-Leu(6) sodium salt by dissolving 5 g of polymer into 100 mL of saturated NaHCO₃ solution and dialyzed (MWCO=3.5 KDa) against DI water. Freeze-dried polymer was recovered in about 50% yield as white fluffy powder and was characterized by ¹H-NMR (FIG. 1). Elemental Analysis: C₂₈H₄₆N₄Na₂O₁₀ (644.67); Calcd.: C, 49.03; H, 7.83; N, 8.27. Found: C, 52.17; H, 7.19; N, 8.69. Mw=106,000 g/mol, Mw/Mn=1.42 (Size exclusion chromatography (SEC) 10 mM PB S, pH 8.4, OEG standards); Tg=146° C.

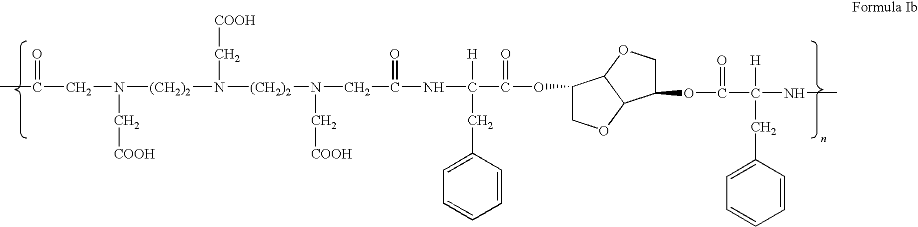

### Polymer Synthesis of PEA DTPA-Phe(DAS), (Formula Ib)

For solution polycondensation, L-Phe(DAS).2TosOH (18.80 mmol, 14.757 g) and DTPA-DA (18.80 mmol, 6.718 g) were mixed together followed by the addition of 15.67 mL of dry DMSO and 11.0 mL of triethylamine (TEA) under argon. The reaction was stirred at room temperature for 24 h and the polymer product was precipitated in 2.5 L of acetone. The supernatant was decanted and polymer was rinsed with acetone and then allowed to air dry. The polymer Formula Ib was re-suspended in DMSO, diluted with 1:1 v/v DI water, transferred into dialysis bags (MWCO=3.5K) and dialyzed in DI water. Dialyzed samples were lyophilized to obtain about a 90% yield of white polymer powder. The weight average Mw=24,500 (g/mol) (GPC), Tg=122° C.

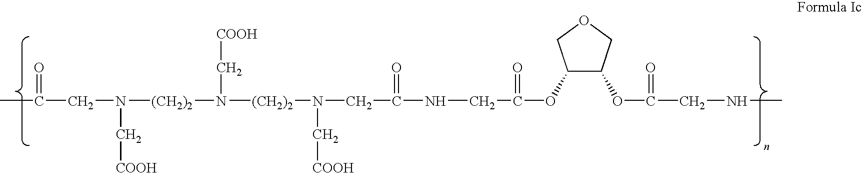

### Polymer Synthesis of PEA DTPA-Gly(THF), (Formula Ic)

For the polycondensation reaction, Gly(THF)-2TosOH monomer (26.06 mmol, 14.664 g) and DTPA-DA (26.06 mmol, 9.313 g) were mixed in 21.72 mL of dry dimethylsulfoxide (DMSO) at room temperature under argon and 15.26 mL of triethylamine (TEA) was added. The polycondensation reaction was continued for 26 hrs and the polymer products were precipitated in 2.5 L of acetone. The supernatant was decanted and polymer was rinsed with acetone and then allowed to air dry. The polymer was re-suspended in distilled H₂O, the solution transferred into dialysis bags (MWCO=3.5K) and dialyzed in distilled H₂O for 3 days (DI water), then lyophilized, to obtain about a 50% yield of a white powdery material. Product Formula Ic was then characterized by ¹H-NMR and SEC. Mw=14,400 g/mol, Mw/Mn=1.62 (SEC, 10 mM PBS, pH 8.4, OEG standards).

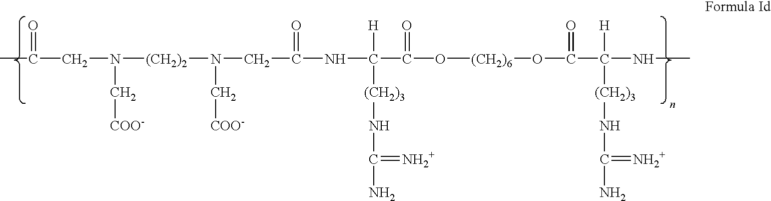

### Polymer Synthesis of PEA EDTA-Arg(6), (formula Id)

A polycondensation reaction was conducted for 1 hr at 45° C. similarly as in preparation of Formulas Ia, Ib and Ic. Then the temperature was increased to 65° C. for another 1 hr to allow complete dissolution of reactants and then stirring was continued again at 45° C. for additional 6 hrs. Polymer was precipitated in acetone, filtered through filter paper and dried in vacuum oven over night. Polymer was redissolved in water along with NaHCO₃, (0.5 g bicarbonate per 5 g of polymer) dialyzed in DI water for 3 days and dried on lyophilizer. No p-toluenesulfonic counter-ion was detected in ¹H-NMR analysis of polymer Formula Id. El. Analysis C₂₈H₅₂N₁₀O₁₀ (688.77); Calcd.: C, 48.83; H, 7.61; N, 20.34. Found: C, 44.95, H, 7.79, N, 18.76. Mw=17,800 g/mol. (SEC).

Size exclusion chromatography (SEC) was used to characterize Mw of the polymer. The instrumentation consisted of a Waters 600 LC pump, a Waters 717 plus autosampler, and a Waters 410 refractive index detector with an internal temperature setting of 30° C. A 50 μL aliquot of the sample solution was injected on to a Waters, Ultrahydrogel® 500, 7.8×300 mm column that was maintained at 30° C. and eluted at 0.6 mL/min using a 100 mM ammonium acetate buffer solution at pH 4.8. A 2.0 mg/mL sample of the PEA-EDTA-Arg(6) polymer was dissolved in 100 mM ammonium acetate buffer, pH 4.8. The retention time of polymer was compared to the retention times obtained from a protein standard (Phenomonex, Aqueous SEC 1) containing a mixture of human thyroglobulin (660 kDa), bovine γ-globulin (158 kDa), ovalbumin (45 kDa), myoglobin (17.8 kla), and uridine (0.48 kDa).

### Synthesis of PEA EDTA Leu(PEG₂₀₀), of Formula Ie

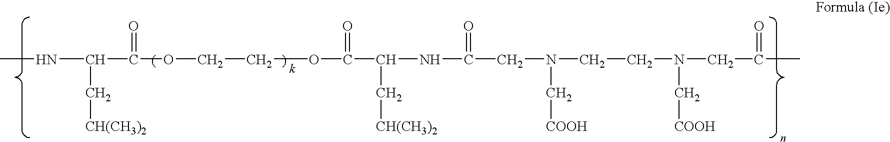

Bis-(L-leucyl)-PEG₂₀₀-diester ditosylate (L-Leu(PEG₂₀₀).2TosOH) (3.177 g) and EDTA-DA (1.0321 g) were mixed together followed by the addition of 2.12 mL of dry N,N-dimethylacetamide (DMF) and 1.24 mL of dry triethylamine (TEA) under nitrogen. The reaction was stirred at 0° C. (ice bath) for 6 h, at room temperature for additional 18 hrs and quenched by the addition of EDTA-DA (0.26 g). Stirring continued for additional 16 hrs at room temperature and the polymer was precipitated in 1 L of acetone. Product was again rinsed with acetone, allowed to air dry, then re-dissolved in 10 mL of saturated NaHCO₃, diluted with 20 mL deionized water, and dialyzed (MWCO=3.5 KDa) against DI water. Freeze-dried polymer was recovered in 2 g yield as white fluffy powder and characterized by ¹H-NMR. (D₂O, ppm, δ): 0.89 [d, d 12H, —CH—(CH₃)₂], 1.60 [m, 4H, —CH—CH₂—CH—(CH₃)₂], 1.74 [m, 2H, —CH—(CH₃)₂], 2.86 [s, 4H, —N—CH₂—CH₂—N—], 3.28 [s, 4H, —NH—CO—CH₂—N<], 3.43 [s, 4H, >N—CH₂—COOH), 3.70-3.78 [m, ˜14H, —O—CH₂—CH₂—O—], 4.26-4.33 [m,m, 4H, —OCO—CH₂—CH₂—O—], 4.47 [m, 2H, —HN—CH<]. Mw=33,000 g/mol, Mw/Mn=1.04; (SEC, 10 mM PBS pH 8.4, +20% v/v MeOH, OEG standards).

### Preparation of Polymer Metal Conjugates and Determination of Binding Capacity

Water Soluble Polymer PEA-DTPA-Leu(6) Complexation with Gd(III):

300 mg of PEA-DTPA-Leu(6)-Na salt (Mw 13,100 g/mol, GPC, DMAc, PS) was dissolved in about 8 mL of DI water. Then an equimolar amount of an aqueous solution of GdCl₃.6H₂O was added drop wise to the solution while stirring. The pH was maintained at 5.8 by the addition of 0.1 M NaOH. Stirring was continued for 1 day. The solution was dialyzed until free Gd ions were no longer detected in the solution (xylenol orange test as described by Barge, A. et al. Contrast Med. Mol. Imaging. (2006) 1:184-188) and then sample was lyophilized. A reduction in the apparent molecular weight values of polymer was observed after complexation (Mw=8,700 g/mol), which result should be attributed to neutralization of charge in the DTPA polymers when bound to metal. Metal binding further caused changes in hydrodynamic values. Content of bound Gd(III) was >90% per DTPA cage, as determined by ICP-MS measurement.

### Example 2

### EGTA Based PEA Synthesis [CO-EGTA]: One-Pot Reaction (Scheme 1)

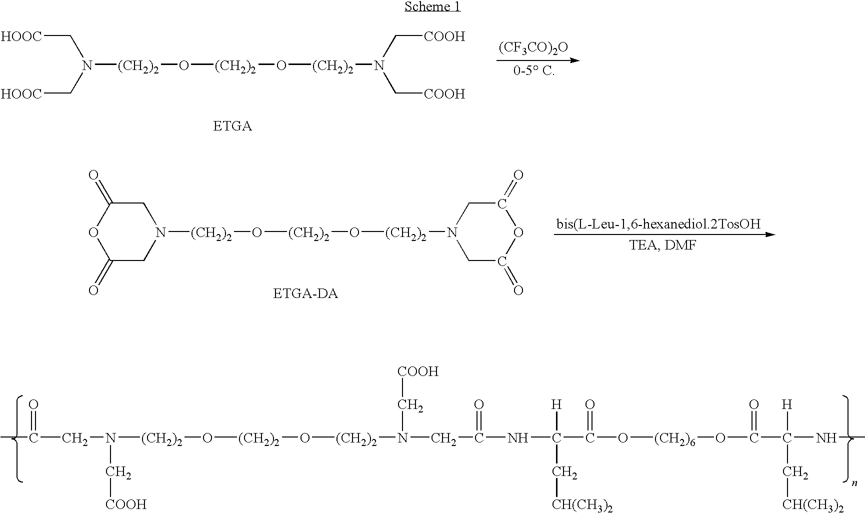

30 mL dry dichloromethane (DCM) and 5 g (13.1 mmol) of ethylene glycol-bis(2-aminoethylether)-N,N,N′,N′-tetraacetic acid (EGTA) were charged into a 250 mL three neck round bottom flask, cooled down on ice bath and blanketed under argon. Then, 4.55 mL (33 mmol) of trifluoroacetic anhydride was added and stirred until the white solid was completely converted into a transparent, pale yellow, EGTA dianhydride viscous layer (ca. 4 hours). Ice bath then was replaced with methanol/dry ice bath and reaction mixture was cooled down to −40° C. to −30° C. Separately, 16.5 mL (0.118 mol) of triethylamine (TEA) was diluted in 20 mL of dry DMF and added drop-wise into reaction mixture over a 1 hr period and stirring was continued for 30 minutes at about −30° C. Then, 9.048 g (13.1 mmol) of diamine monomer di-p-toluenesulfonic acid salt of bis-(L-leucine)-1,6-hexanediol diester was added and stirred overnight at room temperature. The crude polymer solution had Mw=36 kg/mol, Mw/Mn=1.462, (GPC, DMAc, PS). The reaction solution was rotavaporated to remove volatile DCM, diluted with 20 mL water, and dialyzed against DI water. After freeze-drying, 5.94 g polymer was collected with Mw=30 kg/mol, (SEC, PEO). Polymer was further purified by methanol/ethylacetate re-precipitation. Invention polymer structure was confirmed by ¹H NMR analysis in D₂O.

### Example 3

### Formulation of PEA.EDTA.Leu(6).Nickel [Paclitaxel] Nanoparticles

This experiment was conducted to illustrate the invention procedure for formulation of invention metal-chelating polymers as nanoparticles for delivery of a non-water soluble bioactive agent, Paclitaxel.

Preparation of aqueous polymer stock solution (A): 120 mg amount of invention polymer PEA.EDTA.Leu(6) (Mw=24 kg/mol, Mw/Mn=1.68, of Formula I, where R¹=—CH₂—N(CH₂CO₂H)(CH₂)₂N(CH₂CO₂H)—CH₂—; R³═CH₂CH(CH₃)₂, R⁴═(CH₂)₆) was dissolved in 3 mL of 1-Methyl-2-pyrrolidinone (NMP) at room temperature and added drop wise at a rate of 1 mL/min into 17 mL of aqueous 25 mM N-(2-Hydroxyethyl)piperazine-N′-(2-ethanesulfonic acid) (HEPES) buffer with pH=7.0. The buffer solution was stirred vigorously at room temperature to afford a homogenous polymer solution with 6 mg/mL concentration. Stirring was continued for 15 minutes and then the solution was dialyzed over night against 2 L of 25 mM HEPES buffer containing 150 mM NaCl, at pH=7.0. The dialysis membrane was mixed cellulose (Spectropore™) with a molecular weight cut off (MWCO) of 12-14 kDa. Final polymer recovery after dialysis was 82%, as estimated by amino acid analysis. The amino acid analysis was conducted by hydrolyzing the polymer in 6N-hydrochloric acid under inert atmosphere. The hydrolysate was then derivatized with the fluorophore 6-aminoquinolyl-N-hydrozysuccinimidyl carbamate and then analyzed by reverse phase HPLC.

Preparation of Paclitaxel/NiCl₂ stock solution (B): A solution of 2 mg of Paclitaxel (PTX, LC Labs) in 0.95 mL of NMP at room temperature was prepared by vortex stirring. In a separate vial, 5.16 mg of NiCl₂ (Sigma) was dissolved in 0.2 mL of deionized water. Stock solution (B) of PTX/NiCl₂, containing 2 mg/mL PTX and 1.29 mg/mL NiCl₂ (95% v/v NMP, and 5% v/v H₂O) was generated by adding 0.05 mL of the aqueous NiCl₂ solution to the 0.95 mL of PTX solution as a bolus addition. The mixture was stirred via vortex and designated phase (C).

3.1 Invention method for Preparation of PEA.EDTA.Leu(6)Ni[PTX] nanoparticles: 0.5 mL of PEA.EDTA.Leu(6) stock solution (A) was diluted with 2.5 mL of 25 mM HEPES to generate a 0.1% aqueous polymer solution, designated phase (D). PEA.EDTA.Leu(6)Ni [PTX] nanoparticles were generated during the drop wise addition of 0.25 mL of phase (C) with 0.25 mL/min rate of addition, to 3 mL of phase (D) during stirring at room temperature. The mixture was stirred for an additional 5 min and dialyzed overnight against 2 L of 25 mM HEPES, pH=7.0. The dialysis membrane was mixed cellulose (Spectropore™) with a MWCO of 12-14 kDa. The formed dispersion of nanoparticles had a single modal z-average diameter of 151.1 nm as measured by dynamic light scattering (Malvern Zetasizer), and a zeta potential average of −45.5 mV. Final PTX recovery in the nanoparticles was 56.5 μg/mL as determined via HPLC (ACN/H₂0 USP method), and final polymer recovery was 54% as determined by amino acid analysis.

3.2 Controlprocessfor PEA.EDTA.Leu(6)Ni [PTX] nanoparticle formation excluding PEA stock solution: For purposes of comparison, the procedure described in section 2.1 above for formation of product nanoparticles was repeated, except that use of the 0.5 mL of PEA stock solution was replaced by addition of only 500 microliters of 25 mm HEPES. Using this procedure, crystalline aggregates in sizes from 3 to 300 μm were formed, as determined by optical microscopy using a hemocytometer.

3.3 Control process for synthesis of PEA.EDTA.Leu(6)Ni [PTX] nanoparticle formation excluding NiCl₂: The procedure described in section 2.1 above for formation of product nanoparticles was repeated, except that use of 50 μL of NiCl₂ stock solution was replaced with 50 μL of deionized H₂0 added to 0.95 mL of PTX in NMP. The result following dialysis was formation of crystalline aggregates ranging in size from 10 to 500 μm.

### Example 4

### Formulation of PEA.EDTA.Leu(6).Nickel [PTX]-[6-Histidine Tagged-Green Fluorescent Protein] Nanoparticles

This experiment was conducted to illustrate the invention procedure for formulation of invention nickel chelating polymers as water soluble targeted nanoparticles for simultaneously delivery of both a hydrophobic bioactive agent and a His-tagged targeting protein, such as an antibody, or other known proteinaceous targeting ligand. 6His-tagged GFP, which is a protein, not a peptide, is used to model the procedure for chelating a His-tagged targeting protein to invention metal-chelating polymers for delivery of paclitaxel, a highly hydrophobic drug.

Preparation of aqueous polymer stock solution (A): 40 mg of PEA EDTA-Leu(6), (Mw=25 kDa, Mw/Mn=1.59, GPC, PS) free acid form, was dissolved in 4 mL of 25 mM HEPES buffer at pH=11.2, using an ultrasonication bath. The pH after complete dissolution was 7.4. Targeted PEA concentration was 10 mg/mL, with a final polymer recovery of 92%, determined by amino acid analysis.

Preparation of PTX/NiCl₂ stock solution (B): 0.68 mg of PTX was dissolved in 967 μL of NMP at room temperature. In a separate vessel, 5.16 mg of NiCl₂ (Sigma) was dissolved in 0.2 mL of deionized water using vortex stirring and ultrasonication bath at room temperature. A stock solution (B) of PTX/NiCl₂ was generated by bolus addition of 33 μL of the aqueous NiCl₂ solution to the 967 μL PTX solution. The mixture was vortex stirred and the final stock solution containing 0.68 mg/mL paclitaxel and 0.85 mg/mL of NiCl₂ (97% v/v NMP, and 3% v/v H₂O), was designated phase (C).

Invention method for preparation of PEA.EDTA.Leu(6)Ni [PTX]-[6-Histidine Tagged-Green Fluorescent Protein (6His-GFP)] nanoparticles: 0.1 mL of PEA.EDTA.Leu(6) stock solution (A) was diluted with 3.4 mL of 25 mM HEPES pH=7.0. As a bolus, 1 mg of 6His-GFP in 0.5 mL of Tris Buffered Saline (TBS), pH=7.0, was added. The homogenous mixture formed, designated phase (D) was stirred at room temperature for an additional 5 min. PEA.EDTA.Leu(6)Ni [PTX]-[6His-GFP] nanoparticles were generated by drop wise addition with an addition rate of 0.25 mL/min of 0.25 mL of phase (C) into phase (D) during magnetic stirring at room temperature. Stirring was continued for 5 minutes and the mixture was dialyzed overnight against 500 mL 25 mM HEPES, pH=7.0, in mixed cellulose (Spectropore™) membrane with MWCO of 12-14 kDa. The post-dialysis nanoparticle dispersion had a z-average diameter of 86 nm as determined by dynamic light scattering (Malvern Zetasizer), and a zeta potential average of −37.4 mV. Final PTX recovery in the nanoparticles was 14.9 μg/mL as determined by HPLC (ACN/H₂0 USP method), and final polymer recovery was 96% as determined by amino acid analysis. Final 6His-GFP recovery in the nanoparticles was 49% as measured by GFP fluorescence at 485 excitation, 520 emission (Fluostar Optima).

### Example 5

### Formulation of PEA.EDTA.Leu(6).Nickel [PTX]-[Bovine Serum Albumin (BSA)] Nanoparticles

This experiment was conducted to illustrate the procedure for formulation of invention metal-chelating polymers as nanoparticles for targeted delivery of a bioactive agent, paclitaxel, by a common blood protein, bovine serum albumin.

Preparation of aqueous polymer stock solution (A): 150 mg of PEA.EDTA.Leu(6) (Mw=25 kDa, Mw/Mn=1.59, GPC, PS) as free acid was dissolved in 15 mL of 25 mM HEPES buffer at pH=11.15, via sonication bath. Final pH of the solution, designated solution (A), following complete dissolution was 7.4. The PEA concentration was 10 mg/mL, with a final polymer recovery of 83% as determined by amino acid analysis.

Preparation of PEA.EDTA.Leu(6)Ni [PTX]-[Bovine Serum Albumin (BSA)] nanoparticles: 0.11 mL of PEA.EDTA.Leu(6) stock solution (A) was diluted with 3.8 mL of 25 mM HEPES pH=7.0 and mixed with 1 mg of BSA (Fraction V, Sigma) in 0.1 mL of 25 mM HEPES, pH=7.0 solution. The formed homogeneous mixture, designated phase (B), was stirred for 5 minutes at room temperature. Then a dispersion of PEA.EDTA.Leu(6)Ni [PTX]-[BSA] nanoparticles was generated during the drop wise addition (with addition rate of 0.25 mL/min) of 250 microliters of phase (C), prepared as described in Example 4 above, to phase (B) while stirring at room temperature. The dispersion was dialyzed overnight against 0.5 L 25 mM HEPES, pH=7.0 in mixed cellulose (Spectropore™) with MWCO of 12-14 kDa. The post dialysis dispersion had a single modal z-average diameter of 65.7 nm as determined by dynamic light scattering (Malvern Zetasizer), and a zeta potential average of −29.2 mV. Final paclitaxel recovery in the nanoparticles was 19.6 μg/mL, as determined by HPLC (ACN/H₂0 USP method), and final polymer recovery was 73% as determined by amino acid analysis. Final BSA recovery in the nanoparticles was 73% as determined by amino acid analysis.

### Example 6

### Formulation of PEA.EDTA.Leu(6)Zinc [6-Histidine Tagged—Green Fluorescent Protein] Nanoparticles

This experiment was conducted to illustrate the invention procedure for formulation of zinc chelating polymers as nanoparticles for incorporation of a His-tagged protein.

Preparation of aqueous polymer stock solution (A): 22.6 mg of PEA.EDTA.Leu(6) (Mw=34 kDa, Mw/Mn=1.67, GPC, PS) as a free acid was dissolved in 2.26 mL of 25 mM HEPES buffer at pH=7.0, in a sonication bath. Final solution pH was 7.10 The end concentration of PEA was 10 mg/mL, designated stock solution (A).

Preparation of ZnCl₂ stock solution (B): 100 mg of ZnCl₂ was dissolved in 50 mL of 25 mM HEPES buffer at pH 7. The ZnCl₂ stock concentration was 2 mg/mL. When 1.06 mL of the ZnCl₂ stock solution (B) was added to 3.94 mL of HEPES, pH 7.0, an end concentration of 0.423 mg/mL of ZnCl₂, designated solution (B) was obtained.

6.1 Preparation of PEA.EDTA.Leu(6)Zn-[6His-GFP] nanoparticles (C): A dilution of 850 μL of PEA.EDTA.Leu(6) stock solution (A) in 7.65 mL of 25 mM HEPES, pH=7.0 was prepared to yield a polymer concentration of 1 mg/mL. As a bolus, 1 mg of 6His-GFP in 1 mL of Tris Buffered Saline (TBS), pH=7.0, was added to 2 mL of the diluted PEA stock (A) and a homogenous mixture was stirred at room temperature for 5 minutes. Nanoparticles of PEA.EDTA.Leu(6)Zn-[6His-GFP] were generated by drop-wise addition of 1 mL of ZnCl₂ solution (B) at an addition rate of 0.25 mL/min with magnetic stirring at room temperature. The mixture was stirred for an additional 30 min. Nanoparticles formed in the dispersion (6.1) had a z-average diameter of 31 nm as determined by dynamic light scattering (Malvern Zetasizer). Final 6His-GFP recovery in the nanoparticles was 84% as measured by GFP fluorescence at 485 excitation, 520 emission (Fluostar Optima).

6.2 Preparation of Non-PEA controlformulation of PEA.EDTA.Leu(6)Zn-[6His-GFP]. The above procedure for preparation of formulation (6.1) was repeated, except that the 2 mL of PEA solution (A)was omitted and replaced by 2 mL of 25 mm HEPES, pH 7.0. The resulting formulation was determined to contain crystalline aggregates, but no nanoparticles This experiment shows that the presence of invention metal-chelating polymer is necessary to obtain nanoparticles using the polycondensation method.

6.3 Preparation of Non-ZnCl₂ control formulation of PEA.EDTA.Leu(6)Zn-[6His-GFP] nanoparticle: The procedure for preparation of formulation (6.1) was repeated, except that the 1 mL of ZnCl₂ solution was omitted and replaced by 1 mL of 25 mM HEPES pH 7.0. The resulting dispersion (6.3) had particle sizes ranging in diameter from 9 to 500 nanometers. This experiment shows that the presence of the metal ions in the polycondensation procedure assists in formation of nanoparticles made using the invention polycondensation method.

### Example 7

### Formulation of PEA.EDTA.Leu(6).Nickel [PTX]-[Bovine Serum Albumin (BSA)] Nanoparticles

Preparation of aqueous polymer stock solution (A): 87 mg of PEA EDTA-Leu(6) (Mw=82 kDa, Mw/Mn=1.23, (SEC) as a sodium salt was dissolved in 8.7 mL of 25 mM HEPES buffer at pH=7.0, by vortex stirring. Following dissolution, the sample was filtered through a 0.45 μm GHP (hydrophilic polypropylene) disk filter (Pall Life Sciences). Final pH following filtration was 7.54. Final polymer recovery was 79.8%, as estimated by amino acid analysis.

Preparation of PTX/NiCl₂ stock solution (B): 7.5 mg of PTX was dissolved in 750 μL of NMP at room temperature. In a separate vessel, 4.02 mg of NiCl₂ (Sigma) was dissolved in 0.25 mL of deionized water by vortex stirring and ultrasonication bath at room temperature. A stock solution (B) of PTX/NiCl₂ was generated by bolus addition of 250 μL of the aqueous NiCl₂ solution to the 750 μL PTX solution. The mixture was vortex stirred and the final stock solution of 7.5 mg/mL paclitaxel, and 4.02 mg/mL NiCl₂ (75% v/v NMP, and 25% v/v H₂O), was designated phase (C).

Preparation of PEA EDTA-Leu(6)Ni [PTAX]-[Bovine Serum Albumin (BSA)] nanoparticles: 2.0 mL of PEA.EDTA.Leu(6) stock solution (A) was diluted with 6 mL of 25 mM HEPES pH=7.0 and mixed with 20 mg of BSA (Fraction V, Sigma) in 1.0 mL of 25 mM HEPES solution, pH=7.0. A homogeneous mixture formed, designated phase (D), was stirred for 5 minutes at room temperature. PEA.EDTA.Leu(6)Ni [PTX]-[BSA] nanoparticles were generated during the drop-wise addition of 1000 μl of phase (C), at addition rate of 0.25 mL/min, to 9 mL of phase (D) while stirring at room temperature. The dispersion of nanoparticles was dialyzed overnight (16 h) against 0.5 L 25 mM HEPES, pH=7.0 in mixed cellulose (Spectropore™) with MWCO of 12-14 kDa. Following HEPES dialysis, the sample was further dialyzed against 0.5 L 0.9% NaCl (VWR) in analogous dialysis tubing for 3 h. Nanoparticles in the post dialysis dispersion had a z-average diameter of 118.3 nm as determined by dynamic light scattering (Malvern Zetasizer), and a zeta potential average of −17 mV. Final paclitaxel recovery in the particles was 668 μg/mL as determined by HPLC (ACN/H₂0 USP method), and final polymer recovery was 67% as determined by amino acid analysis. Final BSA recovery was 74% as determined by amino acid analysis. These results are summarized below in Table 5.

TABLE 5
Mole to Mole Ratio of Paclitaxel to BSA in example 7
Paclitaxel MW = 853.9 g/mol
BSA MW = 66,430 g/mol

| | | |
|---|---|---|
| Theoretical Mass of BSA: | 20.0 | mg |
| Theoretical Mol of BSA: | 0.301 | μmol |
| Experimental Mass of BSA (via AAA): | 14.8 | mg |
| Experimental Mol of BSA (from AAA mass): | 0.223 | μmol |
| Theoretical Mass of Paclitaxel: | 7.5 | mg |
| Theoretical Mol of Paclitaxel: | 8.78 | μmol |
| Experimental Mass of Paclitaxel (via HPLC): | 6.68 | mg |
| Experimental Mol of Paclitaxel (from HPLC mass): | 7.82 | μmol |

Theoretical Paclitaxel:BSA mole ratio: (8.78 μmol/0.301 μmol) = 29.2 mole ratio of Paclitaxel to BSA
Experimental Paclitaxel:BSA mole ratio: (7.82 μmol/0.223 μmol) = 35 mole ratio of Paclitaxel to BSA

### Example 8

Invention chelating polymers like PEA EDTA-Leu(6) (Formula Ia) are soluble in aqueous solutions and therefore provide a benign environment for the formulation of sensitive biological molecules that can be otherwise structurally unstable in organic mileu, such as nucleic acids (including RNA), antibody fragments, protein domains, and whole proteins. The capacity of this polymer to use metal to induce condensation allows trapping of formulation components in nano- or microparticles, as well as protein display on the particle surface. This latter feature is useful, among other things, for formulation of putative vaccine antigens for testing Recombinant technology can be used to add a poly(histidine) segment, a “His-tag,” to such protein antigen sequences. Such His-tagged proteins promote tethering of antigens to the chelating polymer via the metal ions and allow the display of naturally folded antigenic sites to the immune system when formulations are administered as vaccines.

His-tagged polypeptides for formulation with invention chelating polymers, such as PEA EDTA-Leu(6), can be produced from any known expression system, such as mammalian tissue culture, baculovirus-infected insect cells, yeast and bacteria. Typical protein purification involves cell lysis with microfluidization, followed by ion exchange chromatography and immobilized metal affinity chromatography (IMAC). Proteins prepared for use as vaccines against infectious diseases, such as influenza, should preserve naturally-folded protein domains so both humoral and cellular immunity can be induced by the immune system of the subject receiving the vaccine. Formulations of His-tagged proteins prepared using the invention chelating polymers and methods can be prepared to incorporate one or more proteins into the polymer particles and then the formulations can be mixed, or the vaccine particles can be administered individually with or without other additives, such as adjuvants or targeting moieties.

Because the naturally occurring conformational state of influenza viral hemagglutinin (HA) is critical for robust protective B cell responses, and protection can be provided by antibodies against all portions of this viral protein, a metal condensation formulation of PEA EDTA-Leu(6) with the portion of the influenza viral HA protein that is naturally exposed on the viral surface (the ectodomain), was produced in baculovirus-infected SF9 cells. The pBac-HAPR8 baculovirus was used at a multiplicity of infection of 1 (MOI=1) to infect SF9 cells in 500 mL of Sf900 II-SFM medium (Invitrogen, San Diego, Calif.) at a density of 1.5×10⁶ cells per milliliter. The infected cells were grown for 48 to 72 hours and harvested by centrifugation. The cell proteins were solubilized by suspension in PBS buffer containing 0.1% Triton X-100 and protease inhibitors and then purified by immobilized metal affinity chromatography (IMAC) using Ni-loaded chelating sepharose (GE). Purified protein was dialyzed against two changes of 50 volumes of 25 mM Tris, pH 8.0, 150 mM NaCl, and filtered through 2 micron filters. Since the hemagglutinin antigens need to preserve their natural folding for effectiveness, The recombinantly produced HA ectodomains were tested for sialic acid binding function by a hemagglutination assay following standard protocols (i.e.,Webster, R; Cox, N and Stohr, K, WHO Animal Influenza Manual, World Health Organization, WHO/CDS/NCS/2002.5). Chicken red blood cells were used in an agglutination assay with A/Puerto Rico/8/34 influenza virus as a control.

The DNA sequence encoding nucleoprotein (NP) from influenza A/Puerto Rico/8/34 (NPPR8, SEQ ID NO:1) was designed to encode amino acids 1 through 498 (Genebank accession number NP_{—}040982) plus a hexa-His-tag. The sequence of NP from influenza A/Vietnam//1203/2004 (NPVN, SEQ ID NO:5) encodes amino acids 1 through 495 (Genebank accession number AAW80720) plus a hexa-His-tag. The carboxy-terminal hexa-histidines were included in the gene cassettes encoding each of these viral NP sequences to aid in purification and polymer loading.

Influenza nucleoprotein (NP) gene cassettes were prepared synthetically from overlapping oligonucleotides and PCR and were subcloned into pET26b (Novagen). The NPPR8 and NPVN expression vectors were transformed into BL21-DE3. The bacteria were grown in selective TB medium (Genessee Scientific) to saturation, and then diluted two-fold with fresh, ice cold medium Protein expression was induced in these cultures at room temperature with 200 μM IPTG. After induction for 4 to 6 hours the bacteria were centrifuged and the obtained pellets were frozen. The NP proteins were purified by IMAC. The bacterial pellets were thawed in phosphate buffered saline, pH 7.4 (PBS), and lysed by sonication. The bacterial lysate was centrifuged at 23,000×g and the supernatant was adjusted to 25 mM imidazole then passed over a chelating sepharose column (GE Healthcare) preloaded with nickel. The loaded column was washed sequentially with fifty column volumes of ice-cold wash buffer 3 (50 mM imidazole, 150 mM NaCl, 0.1% Triton X-114, 25 mM sodium phosphate, pH 7.5), and 20 column volumes of wash buffer 4 (50 mM imidazole, 150 mM NaCl, 25 mM sodium phosphate, pH 7.5). The column-bound HA protein was eluted with 500 mM imidazole, in PBS. Eluted NP proteins were dialyzed against two changes of 100 volumes of PBS. Purified recombinant NPPR8 and NPVN were routinely tested for endotoxin content with chromogenic limulus amoebocyte lysate (LAL) assay (Cambrex) and were repurified with additional IMAC cycles of Triton X-114 washes using a known method. (Reichelt, P., C. Schwarz, and M. Donzeau, “Single step protocol to purify recombinant proteins with low endotoxin contents.” Protein Expr Purif (2006) 46(2):483-8) until protein solutions contained below 1 endotoxin unit/mL.

Formulations of PEA EDTA-Leu(6) with His-tagged purified recombinant influenza proteins were made as follows. A solution of Zn Acetate in citrate saline buffer, pH 7 was slowly dripped into a stirring mixture of hexa-His-tagged HAPR8 ectodomain (SEQ ID NO:2) in 25 mM Tris, 150 mM NaCl, pH 8 and PEA-EDTA-Leu(6) in 25 mM HEPES, pH 8 to yield final concentrations of 1 mg/mL His-tagged HAPR8 ectodomain (SEQ ID NO:2), 1.5 mg/mL PEA-EDTA-Leu(6), and 0.367 mg/mL Zn Acetate. His-tagged NPPR8 (SEQ ID NO:1) formulations where made using the same procedure, but the NPPR8 protein was introduced in 25 mM sodium citrate, 150 mM NaCl, pH 7. The NPPR8-Zn-EDTA-Leu(6) formulation contained final concentrations of 0.465 mg/mL His-tagged NPPR8 (SEQ ID NO:1), 0.233 mg/mL PEA-EDTA-Leu(6), and 0.057 mg/mL Zn Acetate. Metal ion condensates of the PEA chelating polymer and influenza antigens were routinely stored at 4° C. until administration.

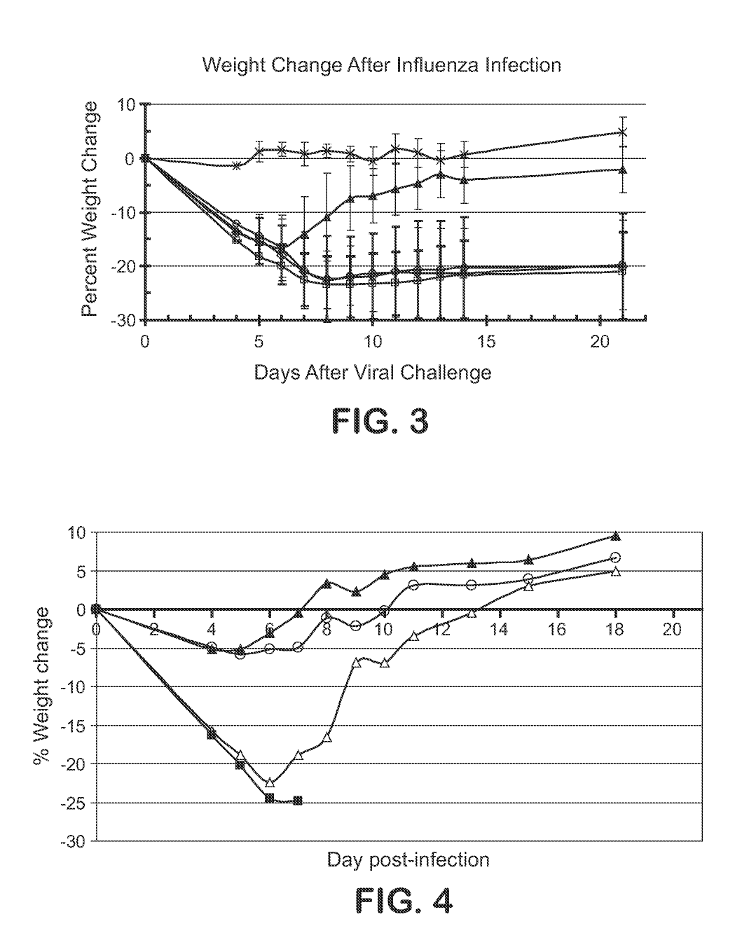

Testing of PEA EDTA-Leu(6)-Zn-Influenza protein antigens was performed by administration to B6/C3 F1 mice. Humoral responses in these animals to both HA and NP antigens were assessed with quantitative ELISAs by evaluating antibodies produced in the serum and bronchiol-aveolar lavages. T cell responses were assessed by measuring interferon gamma via ELISPOT. Interferon gamma production was assessed from splenocytes isolated from immunized mice that had been restimulated with peptides from HA or NP. FIGS. 2 and 3 display data from an experiment in which mice were intranasally administered 1 dose of PEA-EDTA-Leu(6) formulations containing 25 μg of HAPR8-3 and 9 μg of NPPR8. These mice were bled at day 14, and challenged at day 21 intranasally with 10 LD₅₀ of infectious virus. For the next three weeks animal morbidity and mortality were monitored. In FIG. 2 the data show that animals administered a single dose of influenza proteins formulated with zinc and PEA EDTA-Leu(6) did not survive unless this formulation also contained the adjuvant Poly I:C. Although there was significant weight loss in these surviving animals (Fig. II), mice receiving the formulation containing Poly I:C adjuvant survived viral challenge after only one administration of the invention vaccine. This survival correlates with ELISA data showing that anti-HA IgG2a antibodies at the 100 ng/mL level or greater were produced only by the group receiving intraperitoneal viral administration and the group receiving formulations of HAPR8 ectodomain and NPPR8 formulated with PEA EDTA-Leu(6)-Zn with Poly I:C adjuvant. All animals receiving formulations containing formulated NPPR8 protein produced high levels of anti-NP antibodies

### Example 9

In this study both baculovirus-produced and bacterially-produced hemagglutinin (HA) domains that possess agglutination capability are used as putative influenza antigens. The hemagglutination assay described above was used in conjunction with an agglutination inhibition assay in evaluation of formulation candidates. If the HA protein or protein subdomain tested possessed target binding activity before formulation into an invention vaccine the His-tagged HA-formulated with cations such as Zn²⁺, Mn²⁺ or Ni²⁺ must also possess hemagglutination activity. Example influenza hemagglutinin antigen fragments (SEQ ID NOS: 2, 3, 4, 6, 7, 8) or similar sequence fragments from other influenza HA proteins) can be expressed with or without bacterial signal sequences (which are underlined in SEQ ID NOS:3, 4, 7, and 8) depending upon the organism used for production. Purified proteins that pass this hemagglutination test serve as good influenza antigens.

Influenza vaccines have also been tested wherein all protein components of the successful vaccine PEA-EDTA-Leu(6)-Zn formulations were purified from bacteria. In the immunization experiment described below, formulations were supplemented with Poly I:C as an adjuvant, and additional NPPR8 is contained in the formulations compared to the vaccine candidate described in the previous example. In addition, this study tested a prime-boost regimen in an effort to eliminate the morbidity of vaccinated animals after infection.

Formulations of PEA EDTA-Leu(6) (formula Ia) and bacterial expressed His-tagged HA polypeptide, for example HAPR8-3 (SEQ ID NO:4) or HAVN-3 (SEQ ID NO:8), were made as described in Example 8, except that a bacterial signal sequence was included in each sequence. A solution of Zn Acetate in citrate saline buffer pH 7 was slowly dripped into a stirring mixture of His-tagged HA polypeptide in tris saline buffer pH 8 and PEA EDTA-Leu(6) in citrate saline buffer pH 7 sufficient to yield final concentrations of 1.1 mg/mL of His-tagged HA polypeptide, 0.55 mg/mL PEA-EDTA-Leu(6), and 0.120 mg/mL Zn Acetate. NPPR8 (SEQ ID NO:1) formulations for use with bacterial expressed His-tagged HA polypeptide formulations were made as described in Example 8, but at final concentrations of 1.1 mg/mL NPPR8 (SEQ ID NO:1), 0.55 mg/mL PEA EDTA-Leu(6), and 0.12 mg/mL Zn Acetate.

To test the effect of different administration routes for particle formulations of PEA-EDTA-Leu(6) and bacterial expressed His-tagged influenza antigens, the formulations were administered either subcutaneously or intranasally to a group of 10 Balb/c mice. Efficacy of the two administration routes was then compared.

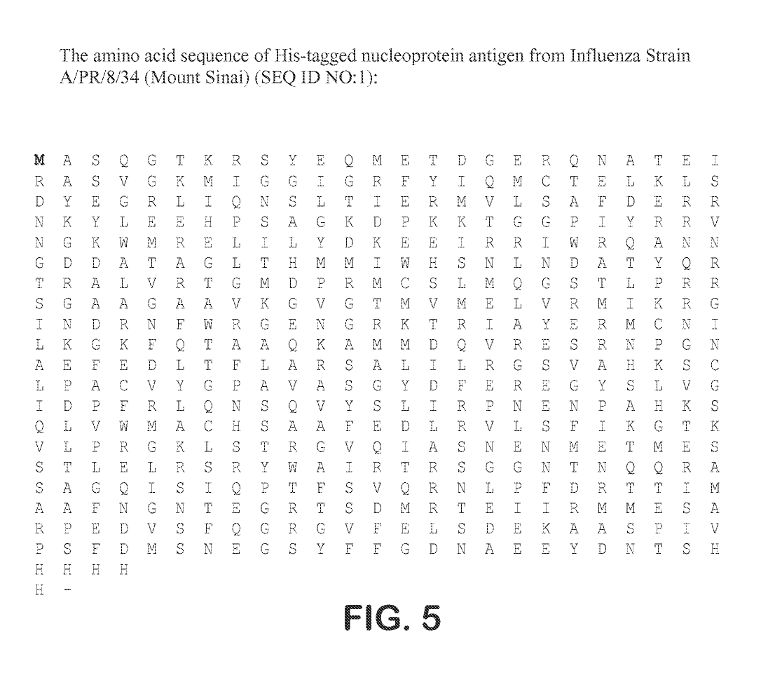

Animals were primed with formulations in which a 50 μl dose contained 25 μg of HAPR8-3 and 25 μg of NPPR8. Each was formulated as PEA EDTA-Leu(6)-Zn particles containing 5 μg of Poly I:C. Two weeks after the first dose, the mice of each group were boosted with a second dose of the same mixture. Three weeks later, all mice were intranasally infected with 10 LD₅₀ of infectious A/Puerto Rico/8/34 virus. The results of these experiments demonstrate the importance of the route of administration for these particulate formulations. For the animals administered the HAPR8-3 and NPPR8 proteins formulated with Zn and PEA EDTA-Leu(6) intranasally, 9 out of 10 animals survived infectious challenge. By contrast, of the animals that were administered an identical formulation subcutaneously, only 1 out of 10 survived. Mice given the intranasal vaccine also exhibited diminished morbidity, as is reflected in the degree of weight loss in response to viral infection illustrated in FIG. 4. These results show that mice vaccinated intranasally had a much better immune response at the same vaccine and dosage than those that were administered the vaccine subcutaneously.

### Example 10

The following conjugation strategies were elaborated for end-group conjugation as depicted in schemes 2 and 3 below:

In the first example of end-group conjugation, an invention PEA chelating polymer was synthesized with predominate amine end groups, and then conjugated with a mono-activated PEG, for example, mPEG-SVA (mPEG-Succinimidyl Valerate, from LaysanBio Inc, Arab, Ala.). The reactions were carried out in aprotic organic solvents (DMSO, NMP), according to scheme 2 below.

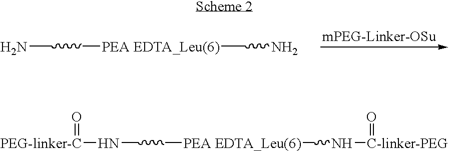

An anhydride end group in the B polymer used also allows for further conjugation of macromolecules or active drugs via amine- or hydroxy-groups, resulting in amide or ester linkages as shown in scheme 3 below.

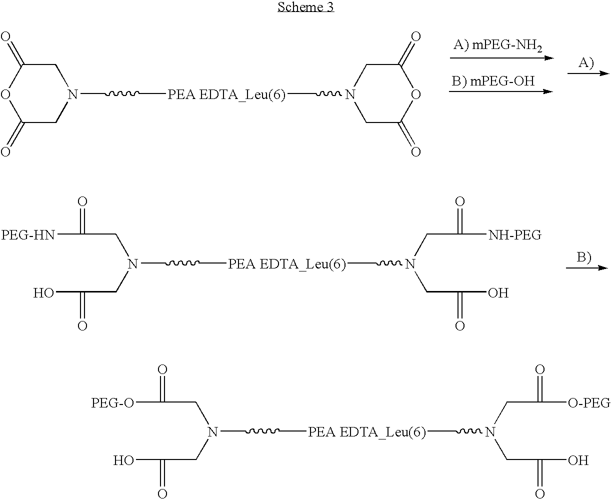

### Synthesis of PEA EDTA-Leu(6) with di-anhydride Ends and Further Conjugation with mPEG-NH₂ to form ABA Block Polymer

5.218 g (7.6 mmol, 0.91 eq) of L-Leu(6)-2TosOH, 2.1326 g (8.3 mol, 1.00 eq) of EDTA-DA were suspended in 2.3 mL anhydrous dimethylsulfoxide (DMSO) and the suspension was blanketed with Argon. Then 4.64 mL (33 mmol) of triethylamine was added and stirring was continued for 3 hours at room temperature. (Mw of crude sample was analyzed by GPC, (DMAc, PS), gave Mw=51,500 g/mol). Then 2.01 g of mPEG-amine (MW 5000, LaysanBio Inc, Arab, Ala.) and 4 mL DMSO were added and stirring was continued over night at 50° C. Polymer was precipitated in 500 mL of acetone, re-dissolved in 100 mL DI water. For complete dissolution of polymer, 15 mg of NaHCO₃ was added, and the solution was dialyzed in MWCO=12-14 KDa dialysis bags against DI water. Freeze-dried polymer was recovered in 2.2 g yield as white fluffy powder and the presence of conjugated PEG was confirmed by ¹H-NMR (MeOD). Mw=36,000 g/mol, Mw/Mn=1.38; (SEC, 10 mM PBS pH 8.4, +20% v/v MeOH, OEG standards.)

Conjugation of PEA EDTA-Leu(6)-dianhydride End Polymer with Laminarin

In a further exemplification, a polysaccharide adjuvant, such as a glucans, was end-group conjugated to the invention chelating polymer. In this example Laminarin, a commercially available representative of the gucans, was utilized as a representative polysaccharide adjuvant useful in vaccine preparation. Conjugation of the adjuvant was accomplished according to Scheme 4 below:

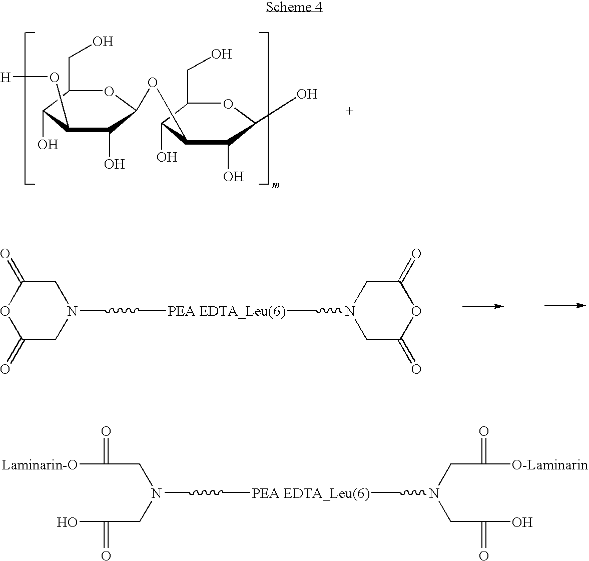

More particularly, 4.283 g (6.2 mmol, 0.84 eq) of L-Leu(6)-2TosOH, 1.8926 g (7.4 mol, 1.00 eq) of EDTA-DA were suspended in 7.95 mL anhydrous N-methyl-2-pyrrolidone (NMP) and blanketed with Argon. Then 1.9 mL (14 mmol) of triethylamine was added and stirring was continued for 16 hours at room temperature. (Mw of crude sample was analyzed by GPC, (DMAc, PS), gave Mw=51,000 g/mol). Separately, 1 g of Laminarin (Aldrich, Mw=5,000 g/mol) was dissolved in 7.5 mL of NMP and 2 mL of polymer reaction solution was added (about 2 mL), then additional 13.9 μL of TEA was added and the solution was stirred at 60° C. for additional 16 h. The solution was diluted with 100 mL DI water, transferred into 12-14 KDa MWCO dialysis bags and dialyzed against DI water. Freeze-dried polymer was recovered in 1.18 g yield as white fluffy powder. Conjugated polymer tested negative in ninhydrin test. The presence of conjugated Laminarin was confirmed by ¹H-NMR (DMSO-d₆) in 37% w/w load. Mw=70,000 g/mol, Mw/Mn=1.2; (SEC, 10 mM PBS pH 8.4, +20% v/v MeOH, OEG standards.).

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention. Although the invention has been described with reference to the above examples, it will be understood that modifications and variations are encompassed within the spirit and scope of the invention.

Accordingly, the invention is limited only by the following claims.

That which is claimed is:

1. A composition comprising at least one of the following polymers or a salt thereof:
a PEA polymer having a chemical formula described by general structural formula (I), 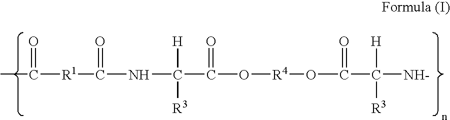
wherein n ranges from about 15 to about 150;
R¹ is —CH₂—N(CH₂CO₂H)—R⁶—N(CH₂CO₂H)—CH₂—, wherein R⁶ is independently selected from the group consisting of (C₂-C₁₂) alkylene, p-C₆H₄, (C₂-C₄) alkyloxy (C₂-C₄)alkylene, CH₂CH₂N(CH₂CO₂H)CH₂CH₂, and a compound having a chemical structure of formula (II), wherein R⁷ is selected from the group consisting of hydrogen, (C₁-C₁₂) alkyl, and a protective group, and combinations thereof; 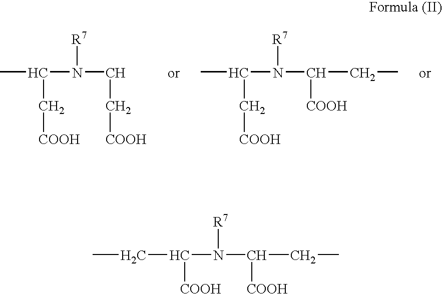
R³s in individual n units are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, —(CH₂)₂SCH₃, CH₂OH, CH(OH)CH₃, (CH₂)₄NH₃⁺, (CH₂)₃NHC(═NH₂⁺)NH₂, 4-methylene imidazolinium, CH₂COO⁻, (CH₂)₂COO⁻ and combinations thereof;
R⁴ is independently selected from (C₂-C₂₀) alkylene, (C₂-C₂₀) alkenylene, (C₂-C₆) alkyloxy (C₂-C₁₂) alkylene, CH₂CH(OH)CH₂, CH₂CH(CH₂OH), a bicyclic-fragment of a 1,4:3,6-dianhydrohexitol of structural formula (III), a fragment of 1,4-anhydroerythritol, and combinations thereof; 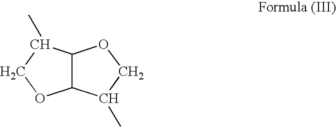
or a PEA polymer having a chemical formula described by structural formula (IV): 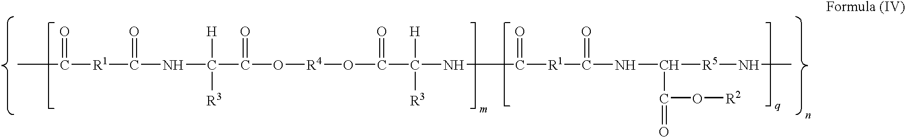
wherein n ranges from about 15 to about 150, m ranges about 0.1 to 0.9; p ranges from about 0.9 to 0.1; and wherein
R¹ is —CH₂—N(CH₂CO₂H)—R⁶—N(CH₂CO₂H)—CH₂—, wherein R⁶ is independently selected from the group consisting of (C₂-C₁₂) alkylene, p-C₆H₄, (C₂-C₄) alkyloxy (C₂-C₄)alkylene, CH₂CH₂N(CH₂CO₂H)CH₂CH₂, and a compound having a chemical structure of formula (II), wherein, R⁷ is selected from hydrogen, (C₁-C₁₂) alkyl, a protective group, and combinations thereof; 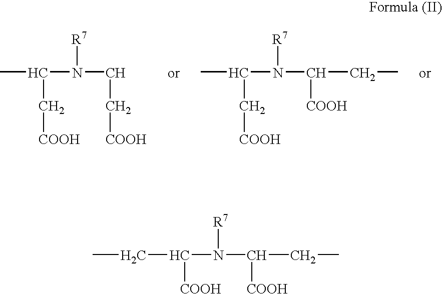
R² is independently selected from the group consisting of hydrogen, (C₁-C₁₂) alkyl or (C₆-C₁₀) aryl and a protective group;
R³s in individual n units are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, —(CH₂)₂SCH₃, CH₂OH, CH(OH)CH₃, (CH₂)₄NH₃⁺, (CH₂)₃NHC(═NH₂⁺)NH₂, 4-methylene imidazolinium, CH₂COO⁻, (CH₂)₂COO⁻ and combinations thereof; R⁴ is independently selected from the group consisting of (C₂-C₂₀) alkylene, (C₂-C₂₀) alkenylene, (C₂-C₆) alkyloxy (C₂-C₁₂) alkylene, CH₂CH(OH)CH₂, CH₂CH(CH₂OH), a bicyclic-fragment of a 1,4:3,6-dianhydrohexitol of structural formula (III), a fragment of 1,4-anhydroerythritol, and combinations thereof; and
R⁵ is independently selected from the group consisting of (C₁-C₄) alkyl.

2. The composition of claim 1, wherein R¹ is —N(CH₂CO₂H)—R⁶—N(CH₂CO₂H)— wherein R⁶ has a chemical structure described by structural Formula (II) wherein R⁷ is selected from the group consisting of hydrogen, (C₁-C₁₂) alkyl, and a protective group.

3. The composition of claim 1 further comprising a metal ion in a complex with the polymer, which metal ion is selected from the group consisting of those of Ca²⁺, Mg²⁺, Mn²⁺, Co²⁺, Fe²⁺, Fe³⁺, Ni²⁺, Zn²⁺ and combinations thereof.

4. The composition of claim 2, further comprising in the complex at least one cargo molecule selected from the group consisting of a polar molecule, a His-tagged molecule, a biologic molecule, and a lipophilic therapeutic molecule with micro-regions of negative polarity consisting of unsaturated regions and/or lone pairs of electrons in an O-, S- or N-containing group, and combinations thereof.

5. The composition of claim 4, wherein the at least one cargo molecule is selected from the group consisting of Paclitaxel, Sirolimus, Everolimus, Docetaxel and Biolimus.

6. The composition of claim 4, wherein the at least one cargo molecule comprises a serum albumin.

7. The composition of claim 4, wherein the at least one cargo molecule comprises a ligand that binds specifically to a target cell, organ or tissue.

8. The composition of claim 4, wherein the at least one cargo molecule is toxic to or binds specifically to a target cell, organ or tissue.

9. The composition of claim 1, further comprising a metal in a complex with the polymer, which metal is selected from the group consisting of those of Gd(III) and radioactive isotopes of Rh, Ir, Yt, and wherein the composition is a diagnostic composition.

10. The composition of claim 9, wherein R¹ is —N(CH₂CO₂H)—R⁶—N(CH₂CO₂H)— wherein R⁶ is CH₂CH₂N(CH₂CO₂H)CH₂CH₂ and the metal is Gd(III)

11. The composition of claim 7, further comprising at least one cell-killing or targeting cargo molecule selected from the group consisting of a polar molecule, a biologic molecule, a His-tagged molecule, and a lipophilic molecule having micro-regions of negative polarity consisting of unsaturated regions and/or lone pairs of electrons in an O-, S- or N-containing group.

12. A method for making nanoparticles, said method comprising:
a) contacting together in an aqueous solution under polycondensation conditions:
1) the at least one polymer of claim 1;
2) a metal ion selected from the group consisting of Ca²⁺, Mg²⁺, Mn²⁺, Co²⁺, Fe²⁺ and Fe³⁺, Zn²⁺, Ni² and Gd³⁺; and
3) an aprotic polar solvent;
b) forming nanoparticles containing a non-covalent complex of the polymer and the metal cation in the solution; and
c) obtaining the nanoparticles from the solution by size exclusion separation.

13. The method of claim 12, wherein the solution further comprises at least one cargo molecule selected from the group consisting of a polar molecule, a biologic molecule, a His-tagged molecule, and a lipophilic molecule with micro-regions of negative polarity consisting of unsaturated regions and/or lone pairs of electrons in O- and S- and N-containing groups and wherein the complex in the formed nanoparticles further comprises the at least one cargo molecule.

14. The method of claim 12, wherein the solution further comprises an amino acid sequence of SEQ. ID NO: 1, 2, 3, 4, 5, 6, 7 or 8.

15. The method of claim 12, wherein the His-tagged molecule comprises an amino acid sequence containing a pathogenic epitope.

16. The method of claim 15, wherein the His-tagged molecule is recombinantly expressed into the solution.

17. The method of claim 15, wherein the His-tagged molecule is recombinantly expressed in a bacterium.

18. A composition comprising:
a) a bioactive agent selected from the group consisting of an oligo- or polyethyleneglycol, a polysaccharide, a lipid, a biologic macromolecule and a water insoluble drug; and
b) a polymer of claim 1,
wherein the composition is a linear polymer in which the polymer is flanked on both sides by the bioactive agent.

19. The composition of claim 18 wherein the bioactive agent is a polymeric immunostimulating adjuvant.

20. The composition of claim 19, further comprising:
c) a metal ion selected from the group consisting of Ca²⁺, Mg²⁺, Mn²⁺, Co²⁺, Fe²⁺ and Fe³⁺, Zn²⁺, Ni²; and which metal ion is held in
d) an amino acid sequence comprising a pathogenic epitope,
wherein the metal ion and the amino acid sequence are attached to the polymer via a non-covalent complex with R¹ of the polymer.


SEQUENCE LISTING (8 sequences)

SEQ ID NO: 1 - protein, 505 aa, Influenza A virus
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDNTSHHHHHH

SEQ ID NO: 2 - protein, 514 aa, Influenza A virus
MKANLLVLLSALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNIYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNTPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEEHHHHHH

SEQ ID NO: 3 - protein, 373 aa, Influenza A virus
MKKNIAFLLASMFVFSIATNAYAMKANLLVLLSALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNIYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNTPSIQSGGHHHHHH

SEQ ID NO: 4 - protein, 254 aa, Influenza A virus
MKKNIAFLLASMFVFSIATNAYAKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNIYQNENAYVSVVTSNYNRRFTPEIAERPKVRDQAGRMNYYWTLLKPGDTIIFEANGNLIAPMYAFALSRGFGSGIITSSGHHHHHH

SEQ ID NO: 5 - protein, 503 aa, Influenza A virus
MASQGTKRSYEQMETGGERQNATEIRASVGRMVSGIGRFYIQMCTELKLSDYEGRLIQNSITIERMVLSAFDERRNRYLEEHPSAGKDPKKTGGPIYRRRDGKWVRELILYDKEEIRRIWRQANNGEDATAGLTHLMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWMACHSAAFEDLRVSSFIRGTRVVPRGQLSTRGVQIASNENMEAMDSNTLELRSRYWAIRTRSGGNTNQQRASAGQISVQPTFSVQRNLPFERATIMAAFTGNTEGRTSDMRTEIIRMMESARPEDVSFQGRGVFELSDEKATNPIVPSFDMNNEGSYFFGDNAEETSHHHHHH

SEQ ID NO: 6 - protein, 517 aa, Influenza A virus
MEKIVLLFAIVSLVKSDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEHHHHHH

SEQ ID NO: 7 - protein, 370 aa, Influenza A virus
MKKNIAFLLASMFVFSIATNAYAMEKIVLLFAIVSLVKSDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQSGGHHHHHH

SEQ ID NO: 8 - protein, 254 aa, Influenza A virus
MKKNIAFLLASMFVFSIATNAYAGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSESGHHHHHH